(19) United States
(12) Patent Application Publication
(10) Pub. No.: US 2023/0371984 A1
(43) Pub. Date: Nov. 23, 2023

(54) SYSTEMS, DEVICES, AND METHODS FOR FACILITATING SPINAL CORRECTION AND FUSION SURGERY

(71) Applicants: Washington University, St. Louis, MO (US); The George Washington University, Washington, DC (US)

(72) Inventors: Eric Leuthardt, St. Louis, MO (US); Guy Genin, St. Louis, MO (US); Peter Brunner, St. Louis, MO (US); Robert Pless, Washington, DC (US); Camilo Molina, St. Louis, MO (US); Carl Hacker, St. Louis, MO (US); Daniel Moran, St. Louis, MO (US); Alicia Repka, St. Louis, MO (US); Halle Lowe, St. Louis, MO (US); Jacob Sandler, St. Louis, MO (US)

(73) Assignees: Washington University, St. Louis, MO (US); The George Washington University, Washington, DC (US)

(21) Appl. No.: 18/319,445

(22) Filed: May 17, 2023

Related U.S. Application Data
(60) Provisional application No. 63/342,997, filed on May 17, 2022.

(51) Int. Cl. A61B 17/58; A61B 34/20; A61B 90/00

(52) U.S. Cl. CPC A61B 17/58; A61B 34/20; A61B 90/39; A61B 2034/2055

(57) ABSTRACT

Among the various aspects of the present disclosure is the provision of devices, systems, and methods for tracking the positions and orientations of at least one bone screw implanted within a surgical site. The disclosed device includes an extension that includes an extension base configured to couple to the bone screw, a marker arm coupled at one end to the extension base in a hinged arrangement, and an optical marker attached to a free end of the marker arm opposite the hinged attachment. The optical marker includes a pair of lenticular arrays in a coplanar arrangement in which the major axes of the lenticular arrays are mutually perpendicular. Each lenticular array is configured to display different hues at different viewing angles. The disclosed method includes transforming a single image of a surgical site containing at least one bone screw and extension into the global positions of each bone screw based on the pixel positions and hues corresponding to the lenticular arrays of each optical marker.

### CROSS-REFERENCE TO RELATED APPLICATIONS

This application claims priority from U.S. Provisional Application Ser. No. 63/342,997 filed on May 17, 2022, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### BACKGROUND

Many spinal corrections and fusions utilize spinal rods and pedicle screws to provide improved spinal stability and strength. These pedicle screws are inserted into vertebrae at each relevant pedicle, a rod is manually bent into the optimal, but practical, shape, and it is secured into the pedicle screws which hold the spine. To achieve this, a rod is usually bent manually by the surgeon in a time-consuming and sometimes grueling process that can lead to suboptimal rod pathing, screw pullouts, mechanical wear on the rod, and intraoperative revisions. The invention of polyaxial screws lowered the degree of accuracy required in bends by accommodating a far larger range of rod-insertion angles, however the use of polyaxial screws did not obviate the arduous manual process of bending itself nor did it aid the surgeon in deciding on a bending path for the rod.

Although at least several software and hardware systems have been developed to assist surgeons in each process associated with the planning and bending of spinal rods to fit a rod path defined by implanted pedicle screws, each system is accompanied by limitations. One existing system, the Bendini Rod Bending System uses an infrared-tracked wand to touch the tulips of pedicle screws, tracking the location of the head using the distal end of the wand. However, the Bendini system does not interface with the proprietary driver shapes or sizes, and therefore often reports incorrect and inconsistent screw locations.

Spinal correction surgeries require precise placement of pedicle screws that connect metal bars to vertebrae. Tracking these screws is critical for the efficient and accurate completion of these surgeries, and is essential for inserting patient-specific rods that connect between pedicle screws and determine the patient's final spine disposition. However, this is challenging because the screws lie out of plain sight of the surgeon, and because their orientations are difficult to track.

The Medtronic Stealth System offers integrated instruments for tracking Medtronic pedicle screws using a large computer-mounted stereo camera system along with a wand to locate pedicle screws through cannulae during minimally invasive surgery (MIS). The camera of the Medtronic system may be obstructed by the surgical team intraoperatively, by surgical tools, fluids, or many other obstacles, necessitating inconvenient adaptations by the surgical team during an already difficult procedure. Another existing system, the 7D Surgical system uses structured light to enable rapid registration of the patient, spine, and surgical tools but also suffers from obstruction, occlusion, or obfuscation.

### SUMMARY

Among the various aspects of the present disclosure is the provision of devices, systems, and methods for tracking the positions and orientations of polyaxial orthopedic fasteners including, but not limited to, pedicle screws during an open surgical procedure. In some aspects, the positions and orientations of the pedicle screws determined using the devices, systems, and methods disclosed herein are used to design rod pathing used in association with a surgical procedure for spinal correction and/or fusion.

Briefly, therefore, the present disclosure is directed to an extension for a bone screw, a trackable bone screw, and a system for tracking the position and orientation of trackable bone screws.

The present teachings include compositions for an extension for a bone screw configured to track a position and orientation of the bone screw. In one aspect, the extension can include an extension base that can include an attachment fitting configured to couple to the bone screw. In another aspect, the extension base can include a hinge fitting. In another aspect, the extension can include a marker arm that can include a hinge end and a marker support at opposite ends of the marker arm, wherein the hinge end is coupled to the hinge fitting to form a hinged joint. In yet another aspect, the extension can include an optical marker attached to the marker support. In another aspect, the optical marker can include a first and second lenticular array that each includes first and second major axes, respectively. In accordance with another aspect, the first and second lenticular arrays can be positioned in a coplanar arrangement on the marker support and the first and second major axes can be oriented perpendicularly. In some embodiments, the attachment fitting can include a threaded end configured to mesh with a corresponding threaded tulip head of the bone screw. In some embodiments, the attachment fitting further comprises a drive fitting projecting downward from the attachment fitting, the drive fitting ending in a drive end configured to mesh with a shank head of the bone screw. In another embodiment, the drive fitting can be further configured to constrain the tulip head of the bone screw to a monoaxial configuration. In other aspects, wherein the tulip head of the bone screw can be constrained to rotate in an azimuthal rotation about a screw axis of the bone screw. In another aspect, the hinged joint can constrain the marker arm to rotate in a polar rotation relative to the screw axis. In yet another aspect, the hinge fitting and hinge end of the marker can include interlocking features to selectively lock the polar rotation of the marker arm to one of at least two predetermined polar angles. In some embodiments, the at least two predetermined polar angles can be selected from 0°, 15°, 30°, 45°, 60°, and 90°, wherein 0° corresponds to the marker arm projecting upward and parallel to the screw axis and 90° corresponds to the marker arm oriented perpendicular to the screw axis. In some embodiments, the predetermined polar angles are selected from 0°, 30°, 60°, and 90°. In accordance with other aspects, the bone screw can be selected from a monoaxial screw, a polyaxial screw, a uniaxial screw, a uniplanar pedicle screw, and a reduction iliac screw. In yet another aspect, the first and second lenticular arrays can be each configured to display a hue that varies with an orientation of each lenticular array relative to a viewer or image-recording device.

The present teachings also include compositions for a trackable bone screw. In one aspect, the trackable bone screw can include the extension described above coupled to a bone screw. In another aspect, the trackable bone screw can be selected from a monoaxial screw, a polyaxial screw, a uniaxial screw, a uniplanar pedicle screw, and a reduction iliac screw.

The present teachings also include a system for tracking the position and orientation of at least one trackable bone screw. In some aspects, the system can include a computing device. In another aspect, the computing device can include at least one processor. In yet another aspect, the at least one processer can be configured to receive an image of a surgical region. In accordance with another aspect, the image can be a plurality of pixels, each pixel comprising a pixel position and a hue. In another aspect, at least one pixel portion of the plurality of pixels can correspond to an optical marker of a trackable bone screw described above. In other aspects, for each of the at least one trackable bone screws, the system can extract, using the computing device, a first pixel portion of the plurality of pixels corresponding to the optical marker. In yet another aspect, the system can transform the first pixel portion into a global position and orientation of the optical marker based on the pixel positions and hues of the first pixel portion. In another aspect, the system can determine a relative displacement of the optical marker from a screw head of the bone screw based on a polar angle, an azimuth angle, and a length of the marker arm of the extension of the bone screw. In another aspect, the system can determine the global position and orientation of the bone screw by combining the relative displacement of the optical marker from the screw head with the global position and orientation of the optical marker. In some embodiments, the first pixel portion can be transformed into the global position and orientation of the optical marker based on a pinhole camera model subject to a group of constraints corresponding to the pixel positions and hues of the first pixel portion. In one aspect, the global position of the first pixel group can be a rotation matrix R and a translation matrix T defining the rotation and translation of the pixels within a camera coordinate system to corresponding objects in a global coordinate system. In another aspect, the rotation matrix R can be obtained by solving the equations:

R\vec{n}ₕᵤₑ_{₁}·\vec{r}₁=0 Eqn. (8);

R\vec{n}ₕᵤₑ_{₂}·\vec{r}₂=0 Eqn. (9); and

R(C₂-C₁)·(\vec{r}₁×r₂)=0 Eqn. (10),

wherein \vec{n}ₕᵤₑ_{₁} and \vec{n}ₕᵤₑ_{₂} are vectors based on the pixel hues corresponding to the first and second lenticular arrays, respectively; \vec{r}₁ and \vec{r}₂ are rays passing from the origin of a camera origin system through portions of the first pixel group corresponding to the first and second lenticular arrays, respectively; and (C₂-C₁) is the global displacement distance between the first and second lenticular arrays. In another aspect, the translation matrix T can be obtained by solving the equations:

(RC₁+T)·\vec{r}₁=\vec{0} Eqn. (12)

(RC₂+T)·\vec{r}₂=\vec{0} Eqn. (13)

wherein C₁ and C₂ are the global positions of the first and second lenticular arrays, respectively. In yet another aspect, \vec{n}ₕᵤₑ_{₁} and \vec{n}ₕᵤₑ_{₁} can be obtained based on the hues of the portions of the first pixel group corresponding to the first and second lenticular arrays, respectively, using at least one predetermined hue response function. In accordance with another aspect, the system can include an imaging device operatively coupled to the computing device. In another aspect, the imaging device can be configured to obtain an image of the surgical region.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a perspective view of a plurality of tracking devices installed on implanted pedicle screws on both sides of several thoracic vertebrae (T4-T8).

FIG. 1B is a ventral view of the tracking devices of FIG. 1A.

FIG. 2A is a lateral view of a tracking device installed on a single implanted pedicle screw on one side of a lumbar vertebra (L2),

FIG. 2B is a ventral view of the tracking device of FIG. 2A.

FIG. 3A is a side view of an extension installed on a pedicle screw.

FIG. 3B is a partial cross-sectional view of the extension of FIG. 3A.

FIG. 3C is a close-up view of an extension base of the extension of FIGS. 3A and 3B.

FIG. 4 is a side view of the tracking device of FIG. 3A with a lenticular array installed at a free end of a marker arm.

FIG. 5A contains schematic diagrams (top row) and ray diagrams (bottom row) illustrating a lenticular array configured to display different colors depending on a viewer's orientation relative to the lenticular array.

FIG. 5B is a schematic diagram illustrating a coordinate system of a lenticular array.

FIG. 5C is a schematic diagram illustrating a hue response function (HRF) characterizing the relation of a displayed color of the lenticular array of FIG. 5B to a viewer's orientation relative to the lenticular array.

FIG. 6 contains a series of color samples corresponding to the displayed color of a lenticular array at different viewing angles.

FIG. 7 contains graphs (top) and corresponding color maps (bottom) summarizing the hue response functions (HRFs) obtained experimentally for two lenticular arrays.

FIG. 8 contains graphs of the experimentally measured HRFs of FIG. 7 (top row) and corresponding modeled HRFs (bottom row) for two lenticular arrays.

FIG. 9 contains a series of images of the displayed colors of the lenticular array oriented at different viewing angles.

FIG. 10 is a schematic drawing illustrating the coordinate systems used to analyze video images of lenticular arrays to determine viewer orientation.

FIG. 11A is a schematic drawing summarizing the spatial relationship of parameters associated with a transformation of an image from a camera coordinate system to an object coordinate system.

FIG. 11B is a schematic drawing summarizing the spatial relationship of additional parameters associated with a transformation of an image from a camera coordinate system to an object coordinate system.

FIG. 11C is a schematic drawing summarizing the spatial relationship of additional parameters associated with a transformation of an image from a camera coordinate system to an object coordinate system.

FIG. 11D is a schematic drawing summarizing the spatial relationship of additional parameters associated with a transformation of an image from a camera coordinate system to an object coordinate system.

FIG. 12 is a cutaway perspective view of a pedicle screw marker in accordance with one aspect of the disclosure.

FIG. 13A is a graph summarizing raw measurements of hue obtained from a first lenticular array at orientations ranging from 45° to 135°.

FIG. 13B is a graph summarizing the one-to-one relationship of the measurements of hue obtained from the lenticular array of FIG. 13A at orientations ranging from 50° to 105°.

FIG. 13C is a graph summarizing a Fourier fit of the data of FIG. 13B.

FIG. 13D is a graph summarizing raw measurements of hue obtained from a second lenticular array at orientations ranging from 45° to 135°.

FIG. 13E is a graph summarizing the one-to-one relationship of the measurements of hue obtained from the lenticular array of FIG. 13D at orientations ranging from 50° to 105°.

FIG. 13F is a graph summarizing a Fourier fit of the data of FIG. 13E.

FIG. 14 contains a series of hue maps comparing the Fourier fit models of FIG. 13C (left) and FIG. 13F (center), with an ideal, linear hue gradient (right).

FIG. 15 is a cross-sectional view of a pedicle screw and extension base in accordance with an aspect of the disclosure.

FIG. 16 is a top-view drawing of the extension and marker arm installed in the tulip head of a bone screw, showing the azimuthal angle defined by the rotation of the tulip head and marker arm about the screw axis.

FIG. 17 is a block diagram illustrating a method of tracking a bone screw in one aspect.

FIG. 18 is a block diagram schematically illustrating a system in accordance with one aspect of the disclosure.

FIG. 19 is a block diagram schematically illustrating a computing device in accordance with one aspect of the disclosure.

FIG. 20 is a block diagram schematically illustrating a remote or user computing device in accordance with one aspect of the disclosure.

FIG. 21 is a block diagram schematically illustrating a server system in accordance with one aspect of the disclosure.

Those of skill in the art will understand that the drawings, described below, are for illustrative purposes only. The drawings are not intended to limit the scope of the present teachings in any way.

### DETAILED DESCRIPTION

Pedicle screw occlusion poses a significant problem during open spinal correction surgery by inhibiting screw visualization and tracking. In various aspects, systems, devices, and methods to facilitate the tracking of the positions and orientation of pedicle screws during open surgery are disclosed. In some aspects, the pedicle screws are tracked using extensions that interface with existing pedicle screws to provide a mounting surface for a trackable marker at a known relative location to the screws. In various aspects, the trackable markers include lenticular arrays capable of providing screw orientation data as well as position based on images of the markers. Images of the trackable markers are used to calculate the positions of the pedicle screws in space, thereby providing accurate screw location data for accurate and precise computer-automated rod bending. As disclosed further in the Examples below, the tracking system described herein was found to have an angular error in orientation within 5° when back-calculating the angle of each marker relative to its major axis. In various aspects, the disclosed extensions include an extension mechanism configured to interface with an existing pedicle screw design to constrain the screw to a monoaxial configuration while maintaining the marker in a known location relative to the screw. In various additional aspects, the disclosed tracking system may additionally refine pose estimation and incorporate multiple extensions and arrays in a single tracking system as described in additional detail herein.

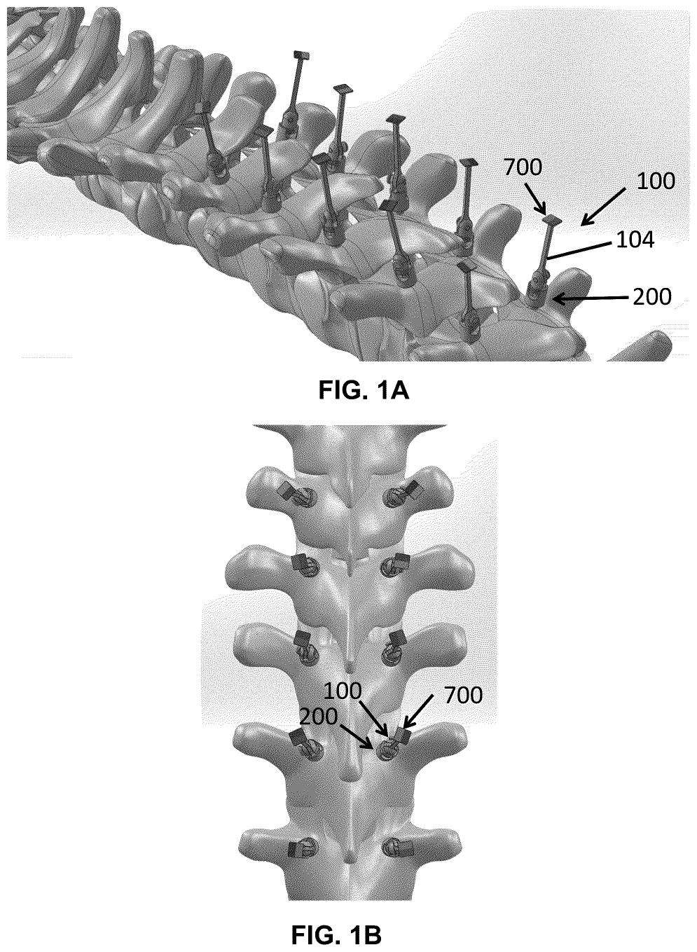

By way of non-limiting example, FIGS. 1A and 1B illustrate pedicle screws 200 fitted with extensions 100 that are provided with optical markers 700. The pedicle screws 200 and extensions 100 are shown implanted into both pedicles of thoracic vertebrae T4-T8. The extensions 100 protrude outward from the pedicle screw 200 in an adjustable direction to ensure that the optical marker 700 is visible to a computer tracking system as described in additional detail below. In this aspect, the pedicle screws 200 are inserted into the thoracic pedicles in an essentially vertical orientation, and the extensions 100 are configured to protrude upward over any layer of fat, tissue, or fluid, and to adjust to maintain visibility in the presence of local obstacles within the surgical site. As illustrated in FIG. 1B, the optical markers 700 attached to the extensions 100 are arranged in a roughly parallel orientation and are easily trackable using a camera system as described herein.

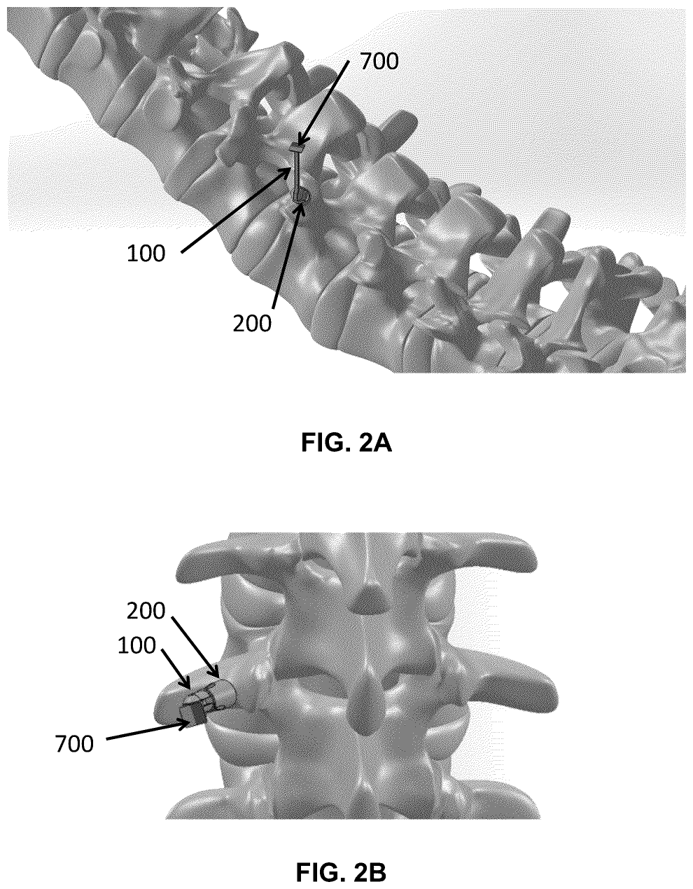

By way of another non-limiting example, FIGS. 2A and 2B illustrate a pedicle screw 200 fitted with an extension 100 and attached optical marker 700 shown implanted into one pedicle of lumbar vertebra L2. Typically, pedicle screws are inserted into lumbar vertebrae at an angle (i.e., in a lateral to medial orientation), rather than vertically as illustrated in FIGS. 1A and 1B. In various aspects, the adjustable orientation of the extension 100 allows for movement of the optical marker 700 to maintain visibility at different orientations of the pedicle screws 200. As illustrated in FIG. 2B, the orientation of the extension 100 allows the optical marker 700 to remain roughly parallel with the thoracic markers in the plane of the image and also avoids occlusion by walls of tissue, fat, or muscle by positioning the optical marker 700 in a more open space.

### Trackable Bone Screw System

In various aspects, a trackable pedicle screw for spinal correction and fusion surgery is disclosed that includes the pedicle screw and an extension reversibly coupled to the tulip or head of the pedicle screw. Each extension is configured to maintain a trackable marker at a predetermined position relative to the pedicle screw to facilitate tracking the spatial position and orientation of each pedicle screw based on images of the trackable marker as described herein. In various aspects, each trackable marker includes at least one lenticular array to provide screw orientation data for the markers in space, which can be used to calculate the positions of the pedicle screws in space, thereby providing accurate screw location data for accurate and precise computer-automated rod bending.

Without being limited to any particular theory, to facilitate the tracking of any type of optical marker after manipulation by surgeons during an operation, either the pedicle screws or a connected device must be clearly visible to a camera system. In various aspects, the optical markers are configured for adjustable positioning within a variety of surgical sites to enhance visibility above the tissue, fluids, and other obstructions within the surgical site from the camera's perspective. In various aspects, the pedicle screw extensions of the trackable pedicle screws are configured to provide an adjustable platform on which the optical marker visibly rests. In some aspects, the extensions are configured to provide at least two degrees of rotational freedom to allow the optical marker to be positioned easily for optimal tracking by the camera. In some aspects, the extensions are configured to interface with existing pedicle screws to maintain a monoaxial configuration, thereby facilitating the determination of a suitable rod path. In other additional aspects, the extensions include adjustably linked rigid elements to provide for the calculation of the position of the screw based on the relevant dimensions of the linked rigid elements of the extension.

a) Bone Screw Extension

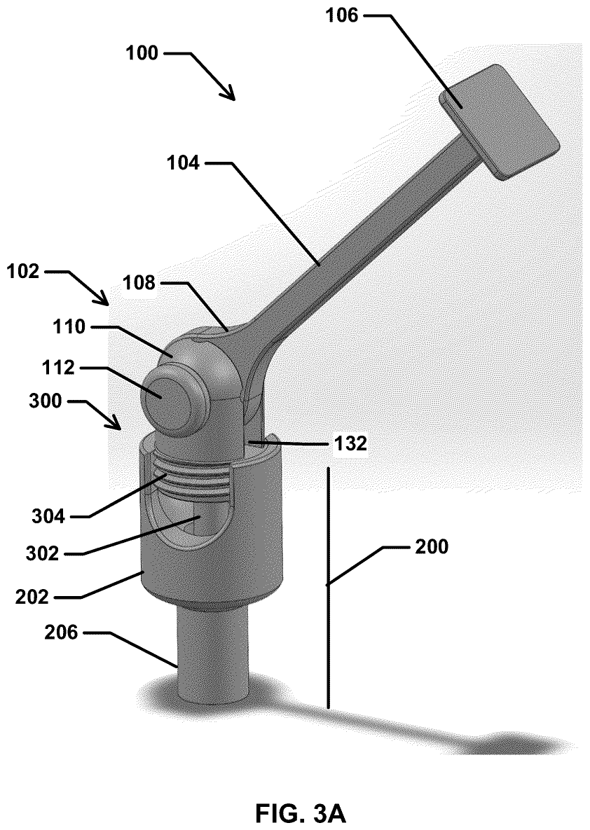

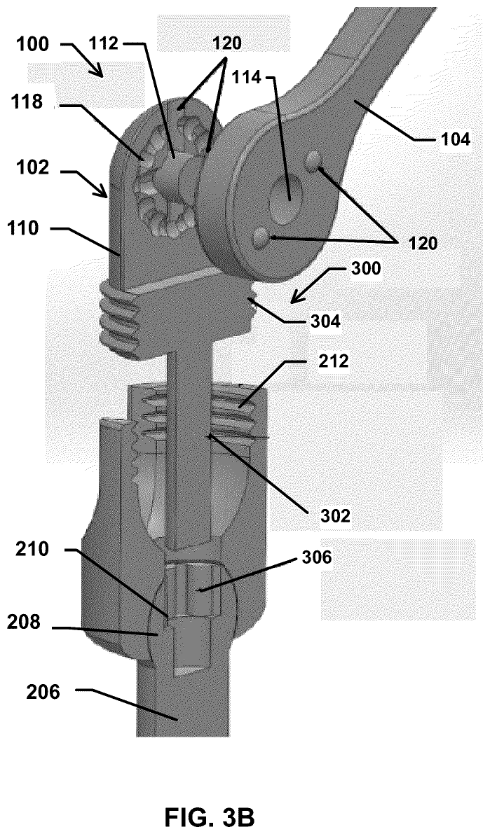

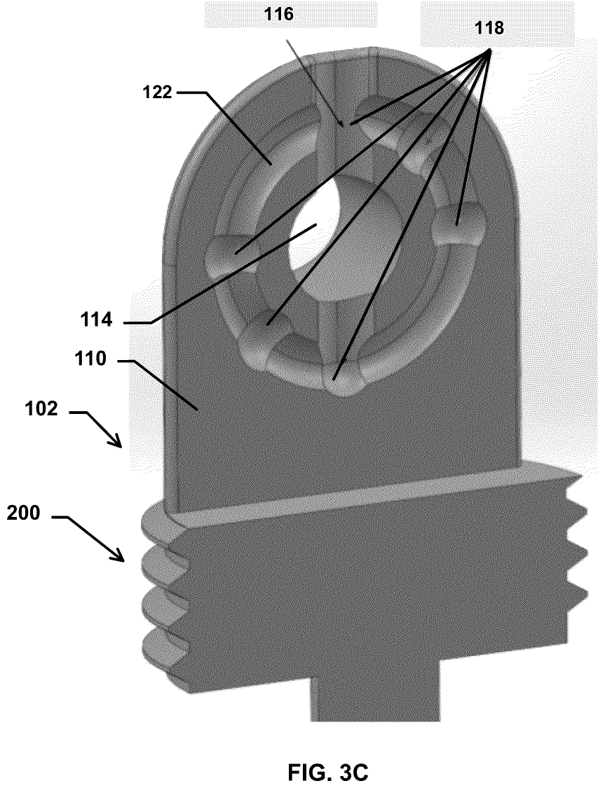

To provide a platform on which an optical marker including, but not limited to, at least one lenticular array, can visibly rest, pedicle screw extensions 100 configured to attach to a pedicle screw 200 or other medical fasteners are included in the disclosed trackable pedicle screws. FIG. 3A illustrates the extension 100 (without optical marker) in one aspect coupled to the head 202 of a pedicle screw 200. The extension 100 is configured to reversibly attach to the head 202 of the pedicle screw 200. The extension 100 includes an adjustable extension arm 104 configured to maintain an optical marker (not shown) in a position suitable for tracking by an imaging-based computer tracking system. In various aspects, the extension 100 includes an extension base 102 configured to thread into a tulip head 206 of a bone screw 200, and an extension arm 104 coupled to the extension base 102 and extending upward.

Referring again to FIG. 3A, the extension base 102 includes a threaded attachment fitting 300 coupled to the tulip head 202 of the pedicle screw 200. The threading 304 of the attachment fitting 300 is sized and dimensioned to mesh with the threaded tulip head 202 of the pedicle screw 200 in one aspect. In various aspects, the threaded attachment fitting 300 is configured to attach to any proprietary fitting of any suitable type of bone screw 200 without limitation. Non-limiting examples of suitable bone screw types include monoaxial pedicle screws, polyaxial pedicle screws, uniaxial pedicle screws, uniplanar pedicle screws, and reduction iliac screws.

Referring to FIGS. 3A and 3B, the extension base 102 further includes a drive fitting 302 projecting downward from the threaded attachment fitting 300 and ending in a drive head 306 configured to mesh with a corresponding drive fitting 210 formed within the shank head 208 of the bone screw 200. In one aspect, shown illustrated in FIG. 3B, the extension base 102 includes threading 212 configured to thread into the proprietary threading 304 of the tulip screw head 202 while locking the bone screw 200 into a monoaxial position by pushing the drive fitting 306 of the monoaxial constraint projection to couple the bone screw 200 and extension 100 in a locked monoaxial configuration.

In various aspects, the extension base 102 further includes a hinged fitting providing a hinged attachment for the extension arm 104, as illustrated in FIG. 3A. Referring to FIGS. 3A and 3B, the hinged attachment is formed by a pair of tangs 110 projecting upward from the extension base 102 to define a gap configured to receive a hinge end 108 of the extension arm 104. An axle 112 extends between the flanges 110 and through the hinge end 108 of the extension arm 104 within the gap 132 to form the hinged joint between the extension arm 104 and the extension base 102.

In various aspects, an axle 112 connects the hinge end 108 of the extension arm 104 between the tangs 110 of the extension base 102 to form the hinge joint. In various aspects, the axle 112 may be formed on at least one or both inward-facing surfaces of the tangs 110, on one or more surfaces of the hinge end 108, or the axle 112 may be provided as a separate element from the tangs 110 and hinge end 108. In some aspects, the axle 112 is defined on at least one inward-facing surface of the tangs 110, and the axle 112 defined in this manner is configured to insert into an axle opening 114 (not shown) formed in the hinge end 108 of the extension arm 104, as illustrated in FIG. 3B. In other aspects, the inward-facing surfaces of the tangs 110 define axle openings 114, as illustrated in FIG. 3C that is configured to receive the axle 112 defined on the surfaces of the hinge end 108. In other additional aspects, the axle 112 may be a separate element inserted through corresponding axle openings 114 formed through the tangs 110 and the hinge end 108 of the marker arm, as illustrated in FIGS. 3A and 3B.

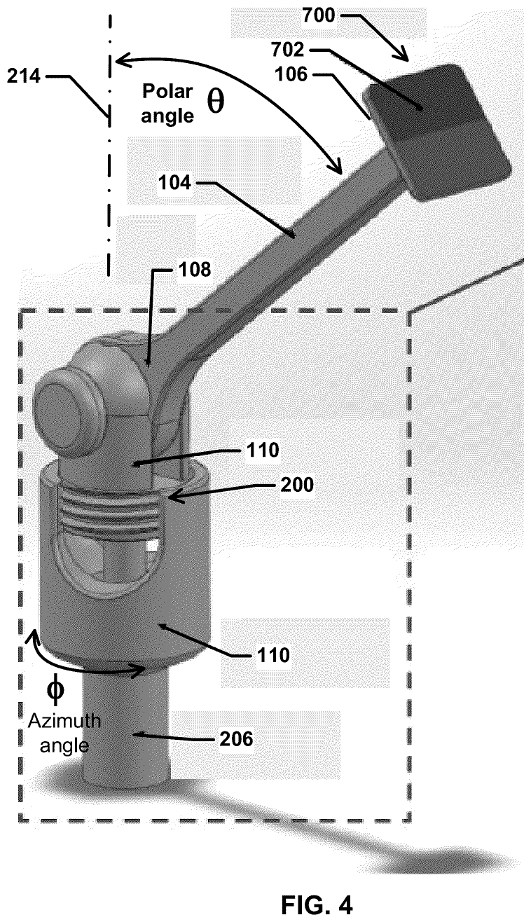

In various other aspects, interlocking features are formed on the adjacent surfaces of the hinge end 108 of the extension arm 104 and the tangs 110. The interlocking features are configured to maintain the extension arm 104 at a polar angle θ relative to the screw axis, as illustrated in FIG. 4. In various aspects, the interlocking features include any known and suitable features configured to lock the marker arm at one or more predefined marker arm angles. Non-limiting examples of suitable interlocking features include circular channels, circular rails, projecting pins or pegs, inverted peg holes or detents, ratcheting teeth, locking screws, and any other suitable interlocking features.

In some aspects, one or both inward-facing surfaces of the tangs 110 may include inverted peg holes 118 and a raised circular rail 122 configured to mesh with a pair of locking pegs 120 formed on the hinge end 108 contact surfaces of the extension arm 104, as illustrated in FIGS. 3B and 3C. In these aspects, the hinge end 108 of the extension arm 104 locks into the extension base 102 using a track of inverted peg holes 118 that provides for the positioning of the extension arm 104 at one of at least several preset polar angles. In some aspects, the pegs 120 of the hinge end 108 may be inserted into the gap 134 between the tangs 110 via an insertion slot 116 formed on the inward-facing surfaces of the tangs 110.

In various aspects, the preset polar angles may be any suitable angle relative to the screw axis 214 (see FIG. 4) without limitation. In some aspects, various aspects, the preset polar angles include at least two or more angles ranging from about 0° (parallel to screw axis 214) and 90° (perpendicular to screw axis 214). In some aspects, the preset polar angles are defined about every 30 degrees, as illustrated in FIGS. 3B and 3C.

In some aspects, the inward-facing surfaces of the tangs 110 may further include one or more insertion slots 116 extending from the outer perimeter to the hinge axis of the tangs. The one or more insertion slots 116 are configured to receive the hinge end 108 of the extension arm 104 after the extension base 102 is fully threaded into the tulip head of the bone screw 204. In various aspects, the insertion slots 116 may be oriented at any suitable predefined angle relative to the screw axis. In one aspect, the insertion slot 116 is oriented vertically along the screw axis, as illustrated in FIG. 3C.

In some aspects, the preset polar angles relative to the screw axis are provided as a set of discrete values that are locked into place by the surgeon in the corresponding peg hole. Although this simplifies communication of the polar angle between the surgical team and the computer system, it also limits the closeness with which the plane of the marker can match the image plane. In other aspects, a more continuous and friction-locking mechanism for choosing a polar angle is included. By way of non-limiting example. both the extender and its associated screw may be made driveable, by introducing successive, detachable sheathes. By introducing a sheath that solely interfaces with the tulip threading while remaining coaxial but rotationally independent from an internal sheath, a stable monoaxial configuration can be achieved for the screw.

In some aspects, the extension 100 may be installed in an assembled configuration that includes the extension base and marker arm coupled in an adjustable hinged arrangement configured to rotate the marker arm to one of the preset polar angles implemented by one of the interlocking features described above. In other aspects, the extension base 102 is threaded into the tulip head of the bone screw without the marker arm, followed by attaching the hinge end of the marker arm to the extension base using the insertion slot described above.

In various aspects, the extension arm 104 further includes a marker support 106 positioned on the free end of the extension arm 104 to the hinge end 108 attached to the extension base 102, as illustrated in FIG. 3A. The marker support 106 provides a mounting surface for an optical marker used to track the position and orientation of the bone screw 200 based on the analysis of images of the optical marker as described in additional detail below. Any suitable optical marker may be attached to the marker support 106 without limitation including, but not limited to, one or more lenticular arrays 702, as illustrated in FIG. 4.

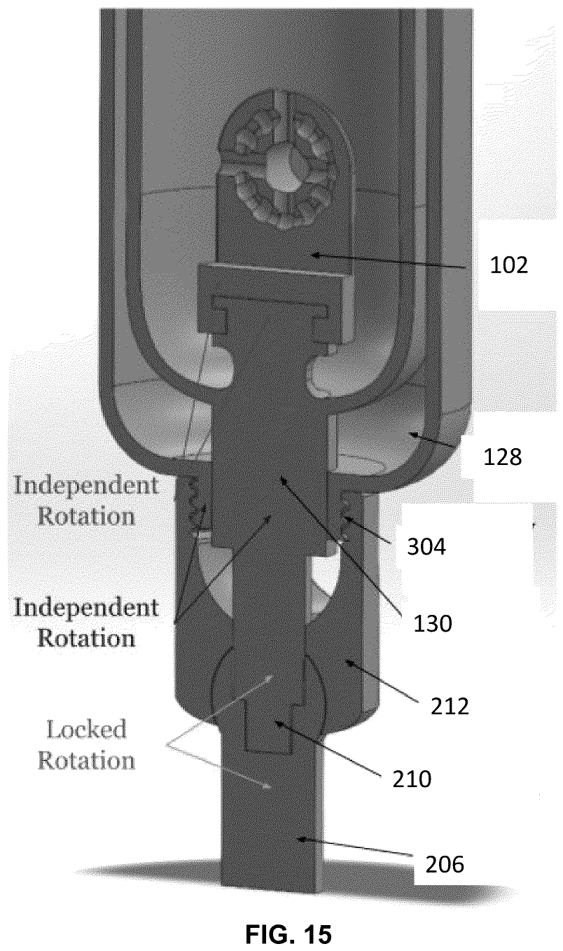

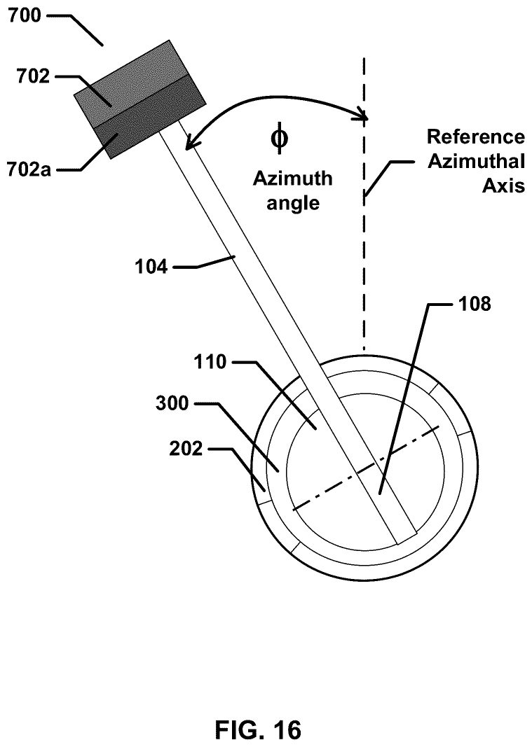

In various aspects, the extension 100 facilitates the positioning of markers 700 to facilitate the imaging and image analysis used to determine the positions and orientations of the bone screws 200 in accordance with the method as described herein. In various aspects, the extension 100 provides for rotation in a polar direction and an azimuthal direction, as illustrated in FIG. 4. Rotation in the polar direction is provided via the hinged joint between the extension base 102 and the extension arm 104. Rotation in the azimuthal direction is provided by rotating the bone screw head 202 as illustrated in FIG. 16. In some aspects, the extension 100 further includes an inner sheath 130, an outer sheath 128, and outer threads 304 on the extension base 102 as illustrated in FIG. 15 to provide for adjustment of azimuthal angle using a driver. Without being limited to any particular theory, the polar and azimuthal rotations position the optical markers of two or more extensions to be positioned roughly parallel between depth planes within an image to facilitate the determination of bone screw positions and orientations as described below.

In various aspects, the optical markers are configured to fully define the extensions orientations and positions in space relative to a camera based on at least one image obtained by the camera. In other aspects, the optical markers are configured to facilitate accurate tracking of the pedicle screws in a variety of light and surgical environments. In various additional aspects, the optical markers are configured to mount within a small surface area of the extension to avoid interrupting the surgery.

Together, these components form a trackable marker with known dimensions and therefore a fully defined equation leading back to the exact location of the pedicle screw, as described in additional detail below.

b) Lenticular Arrays

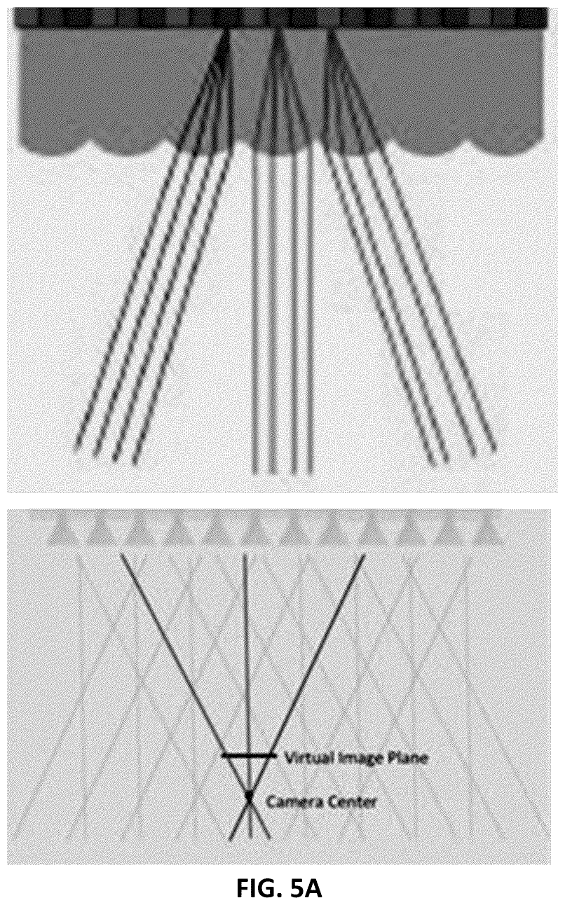

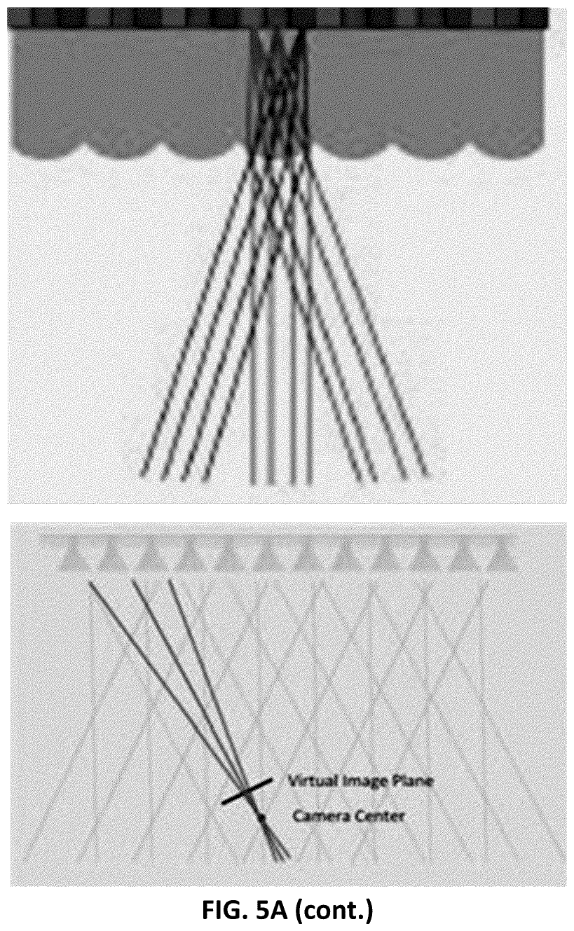

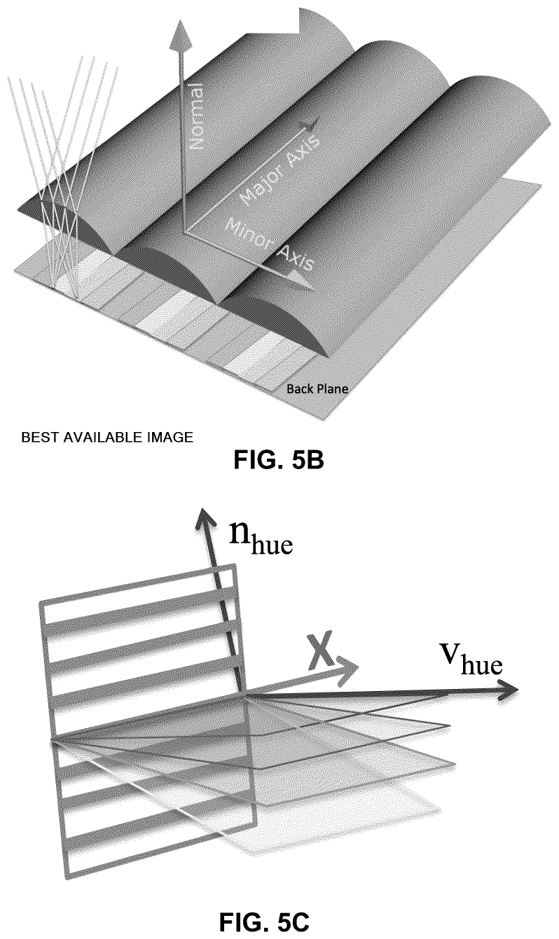

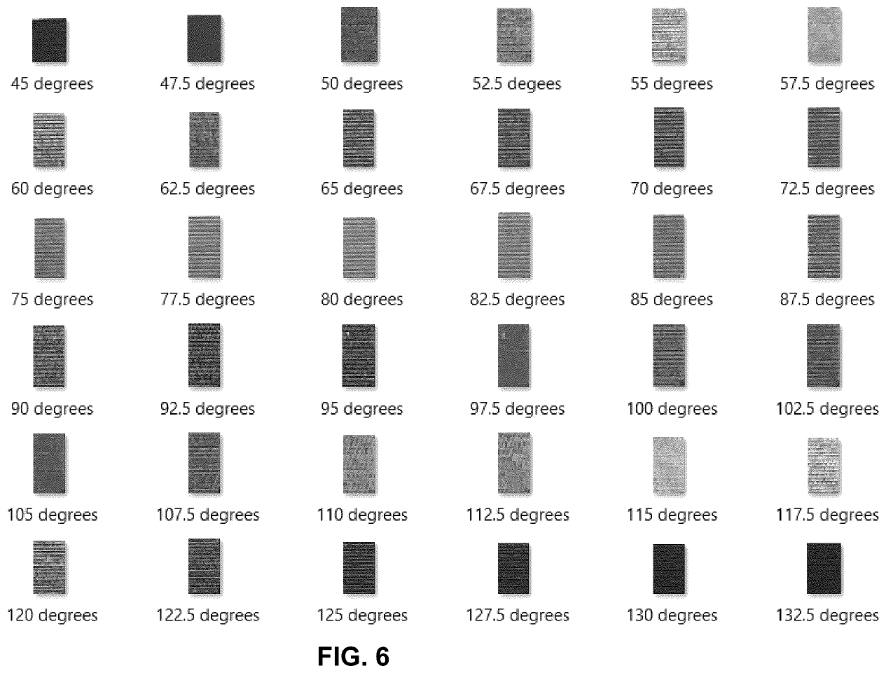

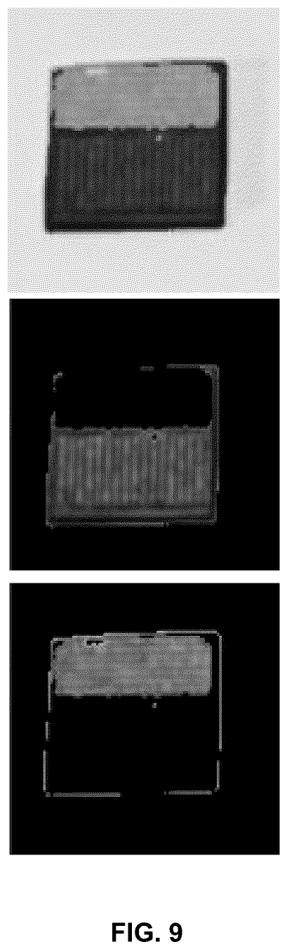

In various aspects, the pedicle screw extensions include lenticular arrays as the optical markers used to define the pose of the extension. Lenticular arrays are sheets of plastic that reflect different colors or patterns when viewed from different directions, as illustrated in FIGS. 6 and 9. This capability comes from the geometry of the array itself, which consists of a backplane lined by parallel cylindrical surfaces, called lenticules, as illustrated in FIG. 5B, reproduced from Schillebeeckx, I., Little, J., Kelly, B., & Pless, R; (2015) The geometry of colorful, lenticular fiducial markers; 2015 International Conference on 3D Vision, the content of which is incorporated by reference in its entirety. The major axis of the lenticular array runs the length of the lenticules and gives the orientation of the array, and the lenticules act as a partial lens focusing parallel planes of light onto the backplane.

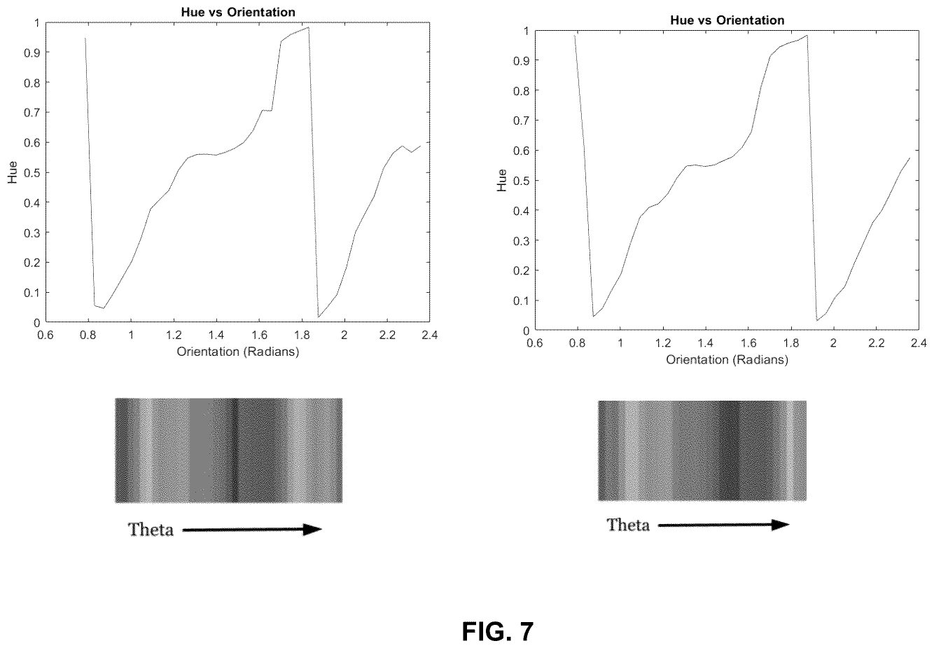

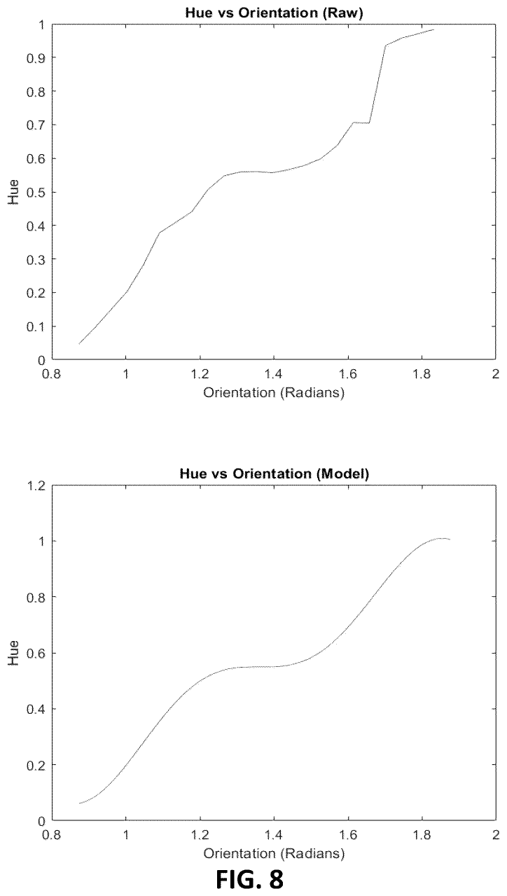

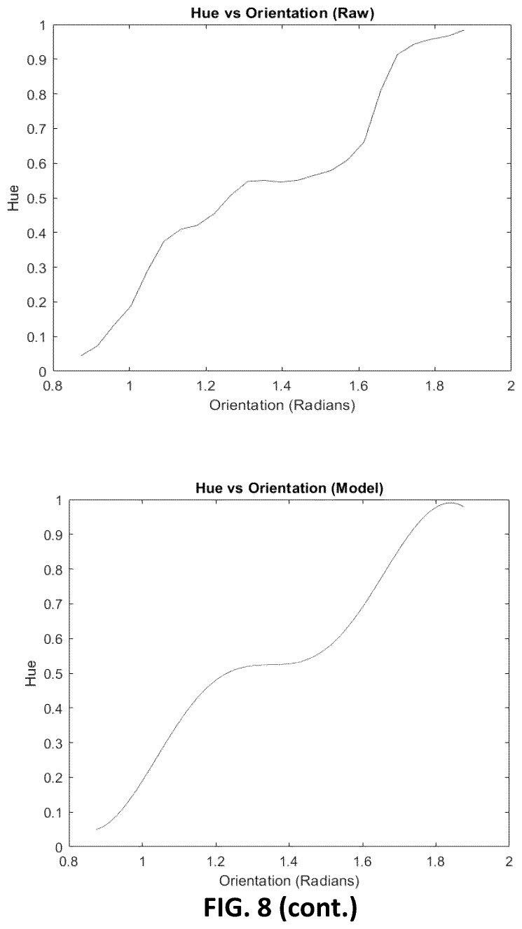

All of the light leaving the lenticular array is reflected from a thin strip behind each lenticule in a direction perpendicular to the lenticule surface, and the color of light observed depends almost exclusively upon the viewing angle, as illustrated in FIGS. 5A and 5C, reproduced from Schillebeeckx (2015). Due to this unique quality, if a continuous rainbow pattern is placed behind the lenticules of a lenticular array, the apparent color of that array will vary smoothly with respect to viewing direction around the major axis. This relationship between the observed hue and the viewing direction is known as the hue response function, or HRF, shown illustrated in one aspect in FIGS. 7 and 8.

Given the strong relationship between hue and orientation, lenticular arrays serve as a practical material for fiducial markers. Using the observed color of a given lenticular array, along with its HRF, it is possible to determine the orientation of the camera about the lenticular array's major axis, and camera pose information can be calculated based on both position and hue of these lenticular fiducial markers (Schillebeeckx et al. 2015). By creating a fiducial marker with two planar lenticular arrays with perpendicular major axes, the orientation and position of the marker in space may be determined using complementary information from each of the arrays, as described below.

Without being limited to any particular theory, experimental lenticular marker-based camera calibration methods have proven to yield a similar surface normal estimation error with a smaller standard deviation when compared with traditional corner-based camera calibration (Schillebeeckx et al. 2015). Additionally, translation estimation error has proven to be significantly lower for lenticular marker-based calibration than for corner-based (Schillebeeckx et al. 2015).

### Method of Tracking Position and Orientation of Surgical Screws

In various aspects, a method of tracking surgical screws is disclosed herein that determines the positions and orientations of at least one surgical screw based on the analysis of images of the optical markers mounted on the screw extensions as described herein. The disclosed method comprises a pose estimation problem that is solved to obtain a rotation matrix R and a translation vector T used to map a point in the object/global coordinate system onto a point in the camera coordinate system. This information, combined with the position and orientation of the tulip head of the bone screw, is used to determine the position and orientation of each bone screw based on an image of the optical markers.

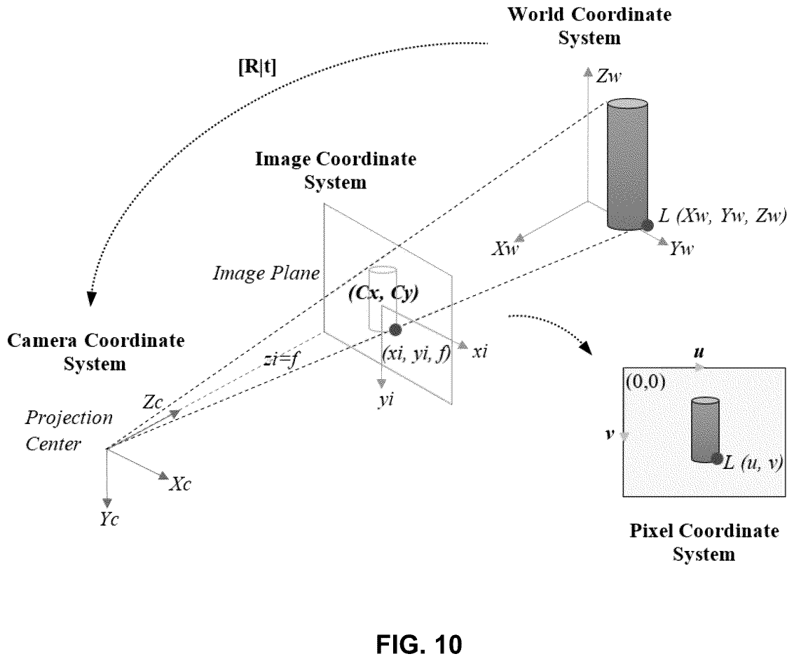

In various aspects, a pinhole camera model is used to define the geometry of the 2D images obtained by a camera. A description of the pinhole camera model is provided in Ortiz, et al. (2017), “A Generic Approach for Error Estimation of Depth Data from (Stereo and RGB-D) 3D Sensors”, the content of which is incorporated by reference herein in its entirety. In brief, FIG. 10, reproduced from Ortiz 2017, provides a schematic overview of a pinhole camera model used to define the geometry of the 2D images obtained by a camera. Referring to FIG. 10, each pixel within an image is coincident with a ray (see dashed lines) connecting the origin of the camera coordinate system with an object or feature of an object within the field of view of the camera. Depending on the focal length of the camera, the image may be positioned at different distances away from the origin of the camera system, but all pixels will be associated with the same objects in the field of view in that they fall somewhere along a ray connecting the origin of the camera coordinate system with the object in the world coordinate system.

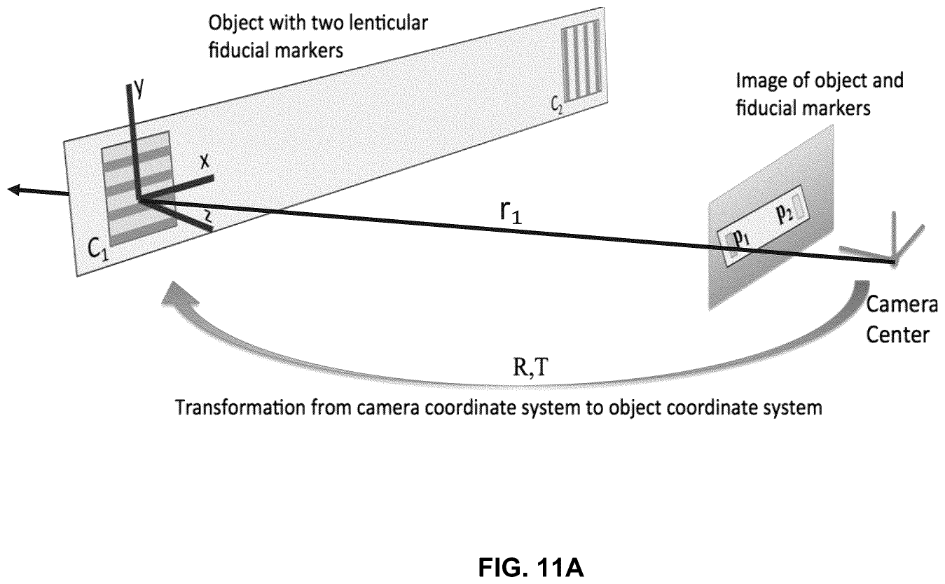

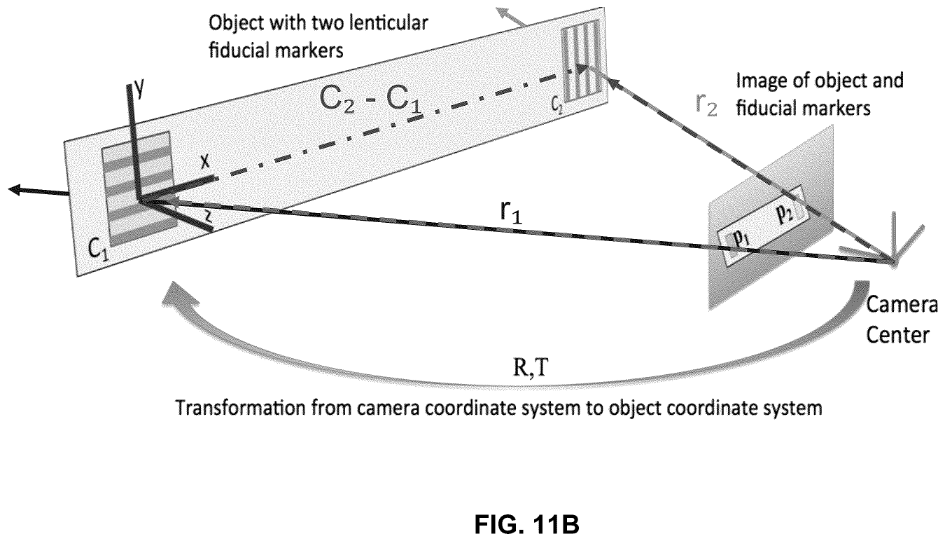

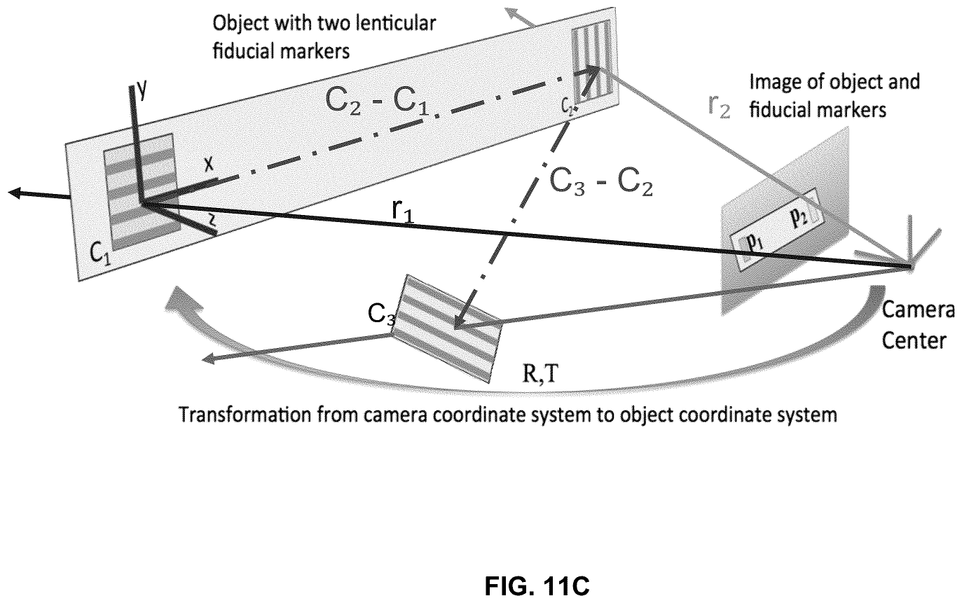

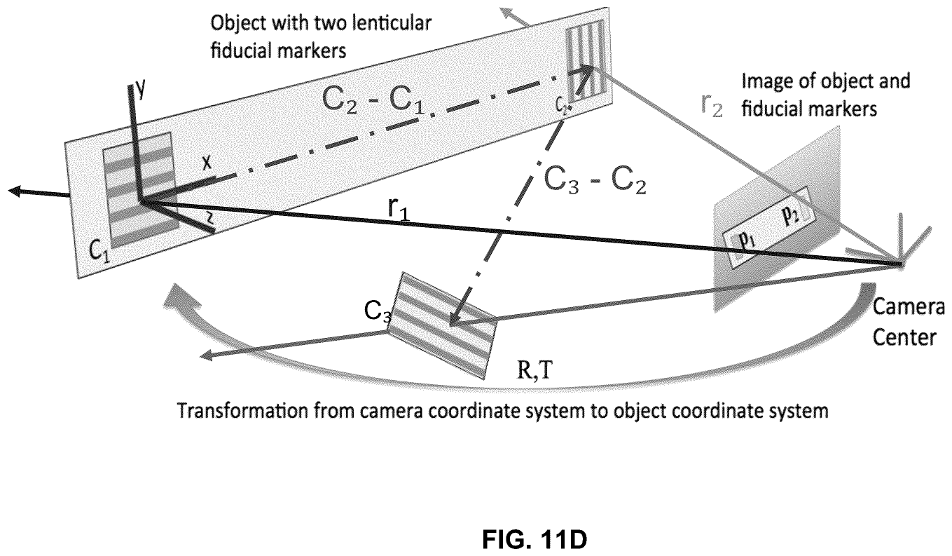

In various aspects, the disclosed method estimates the positions and orientations of the optical markers within a world coordinate system based on camera images. FIG. 11A is a schematic illustration showing the arrangement of the optical markers, image, and pinhole camera model used to obtain the estimated position and orientation of the bone screws. In various aspects, the optical marker includes two lenticular arrays oriented in a coplanar arrangement, in which the major axes of the arrays are mutually perpendicular. The lenticular arrays are positioned within the global coordinate system at positions C₁ and C₂. Based on the hues of the optical markers within the image, corresponding to positions p1 and p2, respectively within the pixel coordinate of the image, as well as the properties of the camera, as defined by the focal length of the camera lens, the position and orientation of the optical marker are determined as described below. Additional description of the method disclosed below is provided in Schillebeeckx, I., Little, J., Kelly, B., & Pless, R; (2015) The geometry of colorful, lenticular fiducial markers; 2015 International Conference on 3D Vision and I. Schillebeeckx and R. Pless (2016); “Single Image Camera Calibration with Lenticular Arrays for Augmented Reality,” 2016 IEEE Conference on Computer Vision and Pattern Recognition (CVPR); 2016, pp. 3290-3298 the contents of which are incorporated herein in their entirety.

According to the minimal constraints detailed by Schillebeeckx et al. 2015, a camera pose can be calculated using two lenticular patches, as illustrated in FIG. 11A. An initial estimate for the camera calibration matrix is given by Eqn. (1):

$$K = ( \begin{matrix}f & 0 & {x}_{0} \\ 0 & f & {y}_{0} \\ 0 & 0 & 1\end{matrix} ) \qquad Eqn . ( 1 )$$

where (x₀; y₀) is the camera center and f is the focal length (Schillebeeckx et al. 2016). Using a pinhole camera model, it can be assumed that the camera origin is at the center of the image and that the calibration matrix is given by K. A pixel p (p₁ in FIG. 11A), represented in homogeneous coordinates, will appear along the ray (r₁ in FIG. 11A) as expressed in Eqn. (2):

\vec{r}=K⁻¹p Eqn. (2)

Thus, lenticular markers at locations p₁ and p₂ will be located along the rays \vec{r}₁ and \vec{r}₂ (FIG. 5C), as expressed in Eqn. (3) and Eqn. (4):

\vec{r}₁=K⁻¹p₁ Eqn. (3)

\vec{r}₂=K⁻¹p₂ Eqn. (4)

Referring to FIG. 5C, since a unique hue corresponds to a unique orientation of a lenticular array, the incident ray must lie in a unique plane, denoted by \vec{n}ₕᵤₑ. \vec{n}ₕᵤₑ is calculated as the cross-product of the major axis \vec{u} (denoted as X in FIG. 5C) and the viewing direction \vec{v}ₕᵤₑ as expressed in Eqn. (5):

\vec{n}ₕᵤₑ=\vec{u}×\vec{v}ₕᵤₑ Eqn. (5)

To simplify calculations, the x- and y-axes of the image coordinate system may be set along the major axes of the first and second lenticular patches, respectively. As illustrated in FIG. 11B, the x and y directions of the image coordinate system are aligned with the major axes of the left and right lenticular arrays, respectively. Therefore the normal vectors for the patches are given by Eqns. (6) and (7):

\vec{n}ₕᵤₑ_{₁}=\vec{x}×\vec{v}ₕᵤₑ_{₁} Eqn. (6)

\vec{n}ₕᵤₑ _{₂}=\vec{y}×\vec{v}ₕᵤₑ_{₂} Eqn. (7)

The ray \vec{r} from the camera that observes each lenticular array lies in the plane defined by \vec{n}ₕᵤₑ, and therefore \vec{r} must be perpendicular to \vec{n}ₕᵤₑ in camera coordinates. Applying this reasoning to rays \vec{r}₁ and \vec{r}₂ yields a pair of constraints as expressed by Eqns. (8) and (9):

R\vec{n}ₕᵤₑ_{₁}·\vec{r}₁=0 Eqn. (8)

R\vec{n}ₕᵤₑ_{₂}·\vec{r}₂=0 Eqn. (9)

It is also known that the two lenticular patches are on a rigid body with a known displacement relative to each other. By way of non-limiting example, the two lenticular patches are mounted on a planar marker support of a bone screw extension, as illustrated in FIG. 4. When translated into camera coordinates, the displacement vector (C₁-C₁) lies within the plane spanned by the viewing rays \vec{r}₁ and \vec{r}₂, as illustrated in FIG. 11B. Consequently, the ray \vec{r}₁ from the camera to the first lenticular patch, the ray \vec{r}₂ from the camera to the second lenticular patch, and the displacement vector R between the first and second patch in camera coordinates are coplanar, which yields a third constraint as expressed in Eqn. (10):

R(C₂-C₁)·(\vec{r}₁×r₂)=0 Eqn. (10)

where C₁ and C₂ are the locations of the markers in world coordinates. Using these first three constraints, the 3×3 rotation matrix R is determined independently from the parameters defining the translation/position of the markers.

Once R has been calculated, additional linear constraints are used to determine a translation matrix T. The translation matrix T is defined to be consistent with the observed locations of the lenticular patches. Consequently, the world coordinates of each marker, once transferred into camera coordinates, must lay on the ray \vec{r} that views each marker. As illustrated in FIG. 11C, any point C_{w} in the world coordinate system is mapped to a camera coordinate location C_{c} as expressed in Eqn. (11):

C_{c}RC_{w}+T Eqn. (11)

Consequently, the ray \vec{r} containing the optical markers at the point C_{c} and the vector from the camera origin to C_{c} must be parallel. Since the cross-product of two parallel vectors is zero, this yields a pair of translational constraints, as expressed in Eqns. (12) and (13):

(RC₁+T)·\vec{r}₁=\vec{0} Eqn. (12)

(RC₂+T)·\vec{r}₂=\vec{0} Eqn. (13)

Since the two translational constraints of Eqns. (12) and (13) are vector equations, the system is fully defined and R and T are calculated by solving Eqns. (8), (9), (10), (12), and (13).

In various aspects, to track each bone screw, an extension as disclosed herein is coupled to the tulip head 202 of the bone screw. In some aspects, the extension contains an optical marker 700 comprising a pair of perpendicularly-oriented lenticular arrays 702 mounted on a fixed-length extension arm 104 as described herein (see FIG. 4). The extension includes various features as described above that provide for the positioning of the marker support at a known distance (the length of the marker arm) and orientation (azimuthal and polar angle) relative to the screw axis. The marker arm length, azimuthal angle, and the polar angle of the marker arm together define the position and orientation of the tulip head relative to the optical marker. In various aspects, the position and orientation of the optical marker obtained from the image are combined with the position and orientation of the tulip head relative to the optical marker to determine the position of the tulip head and orientation of the screw axis. In various aspects, the position and orientation of each bone screw determined in this manner may be communicated to additional software or methods used to visualize, plan, and/or form support rods or other orthopedic appliances used in spinal stabilization procedures.

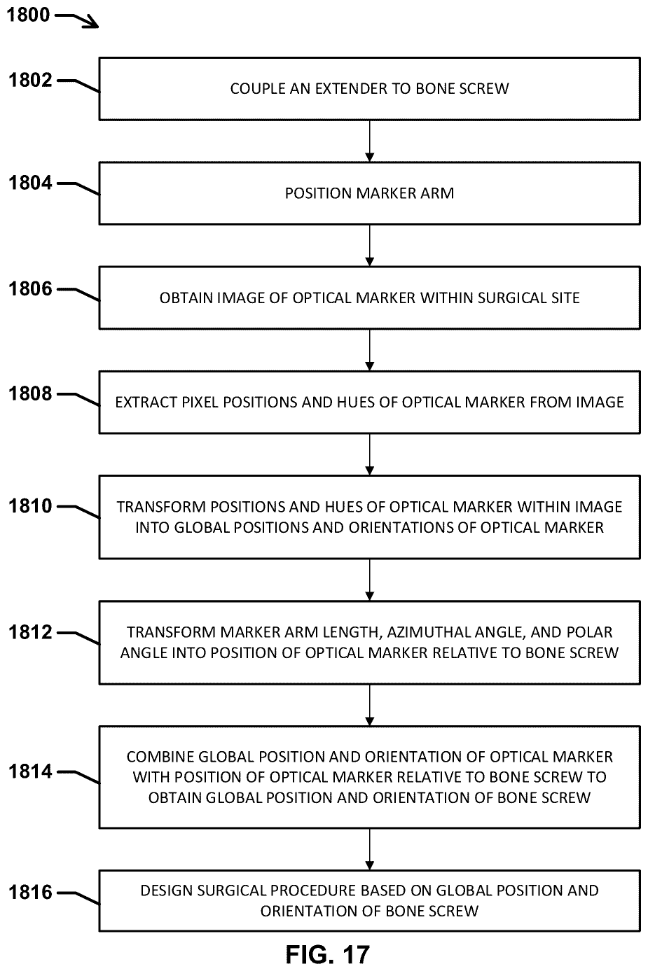

FIG. 17 is a flow chart summarizing a method 1800 of tracking bone screws in one exemplary aspect. The method 1800 includes coupling an extension that includes an optical marker comprising a pair of perpendicularly-oriented lenticular arrays as described herein at 1802. Any suitable extension may be coupled to the bone screw without limitation including, but not limited to any of the extensions with adjustable marker arms as described herein. In various aspects, the extension may be coupled to the bone screw after implanting the screw, the bone screw coupled to the extension may be implanted together, or the bone screw and extension base may be implanted together flowed by attachment of the marker arm, as described herein.

Referring again to FIG. 17, the method 1800 further includes positioning the marker arm of the extension at 1804. In various aspects, the marker arm is positioned by rotating the tulip head and attached extension base to reposition the marker arm at an azimuth angle and by rotating the hinged joint between the marker arm and the extension base to reposition the marker arm at a polar angle as described above. In some aspects, the marker arm is positioned to enhance the visibility of the optical marker by a camera in the presence of occlusions, intervening tissues, or other barriers as described herein. In other aspects, the marker arm is positioned to orient the major axes lenticular arrays of the optical marker within a plane that is relatively parallel to the image plane of the camera. Without being limited to any particular theory, orienting the optical marker in this manner is thought to enhance the accuracy of translations and orientations of the optical marker that are estimated based on images as described above.

In various aspects, the marker arm is positioned at known displacement and orientation to the tulip head of the bone screw. As described above, the extension locks the tulip head in a monoaxial orientation so that the positioning of the marker arm is limited to rotations in the azimuthal and polar directions as described above and illustrated in FIG. 4. Further, the displacement of the optical marker is defined by the fixed length of the marker arm, as described above.

Referring again to FIG. 17, the method 1800 further includes obtaining an image of the optical marker of the extension within the surgical site at 1806. In various aspects, the image includes the one optical marker and may further include additional markers associated with additional bone screws. As described herein, the colors of each lenticular array of each optical marker are indicative of the orientation of the camera relative to the optical marker.

The method further includes extracting the pixel positions and pixel hues of the optical marker at 1808. Any suitable image analysis system or software may be used to extract the pixel positions and pixel hues of the optical marker without limitation. In some aspects, multiple bone screws and attached extensions are imaged at 1806, and the pixel positions and pixel hues of each optical marker are extracted from the image at 1808 and analyzed separately as described below. In various aspects, the regions of the image containing the optical marker are identified and the positions and hues of those pixels are extracted from the image dataset.

The method further includes transforming the positions and hues of the optical markers into global positions and orientations of the optical marker at 1810. As described herein, the optical markers include a pair of lenticular arrays in a coplanar arrangement with the major axes of the arrays oriented mutually perpendicular to one another. As described herein, the color of each lenticular array is indicative of the orientation of the array relative to the image plane of the camera (see FIG. 5C). In various aspects, the orientation of each lenticular array of the optical marker is determined based on a predetermined hue response function (HRF) as described herein. In various additional aspects, the positions and orientations of the optical marker within a global coordinate system are determined using the equations derived from the pinhole camera model as disclosed herein.

Referring again to FIG. 17, the position and orientation of the optical marker relative to the bone screw are determined relative to the optical marker at 1812. As illustrated in FIG. 4, the marker arm is a rigid member of known length that is constrained to move using a combination of changes in azimuthal angle and polar angle. As disclosed herein, the extension provides for these rotations in predetermined and known increments. Consequently, the length of the moment arm, the polar angle, and the azimuth angle are all known quantities that uniquely define the position and orientation of the optical marker relative to the hinge joint of the extension. In various aspects, any suitable known method of translation and rotation of an object based on displacements and rotations may be used to determine the position and orientation of the optical marker relative to the bone screw.

Referring again to FIG. 17, the global position and orientation of the bone screw are determined at 1814. In various aspects, the global position and orientation of the optical marker determined at 1810 are combined with the position and orientation of the optical marker relative to the bone screw determined at 1812 to obtain the global position and orientation of the bone screw. In various aspects, the orientation of the bone screw is provided as the global insertion angle of the screw. In various other aspects, the position of the bone screw is provided as the position of the screw head, the position of the tulip head fitting of the bone screw, or the position of any other suitable element of the bone screw without limitation.

Referring again to FIG. 17, the method may optionally include designing a surgical procedure based on the position and orientation of one or more bone screws at 1816. In some aspects, the positions and orientations of the bone screws may be displayed to a practitioner to inform the planning or implantation of a surgical procedure. In other aspects, the global positions of the bone screws may be used by a variety of existing medical systems and methods for designing a surgical procedure including, but not limited to, designing and/or fabricating reinforcement rods as described herein. In some aspects, the global positions of the bone screws may be communicated directly to an existing system or device using any suitable existing computer data transmitting protocol without limitation.

In various aspects, the tracking of positions and orientations of objects using lenticular arrays goes far beyond the tracking of bone screws using screw extensions as described herein. It is to be noted that lenticular arrays can be used to find pose estimations of any solid body relative to its viewer so long as the constraints disclosed herein are satisfied. Other surgical tools or bodies may be tracked using these markers to provide information about their pose relative to a camera and/or patient. Non-limiting examples of surgical tools suitable for tracking using lenticular arrays include bayonet forceps, drivers, wands, and any other suitable surgical instrument without limitation. The method disclosed herein provides for further development of tracking technologies for visible or extendable implanted devices in addition to bone screws.

In various other aspects, tracking of surgical instruments using the disclosed method to track surgical instruments and devices in association with open spine surgery as well as minimally invasive spine surgery (MISS). In some aspects, the tracking methods described herein may be adapted for use in the positioning and guidance of cannulae that are physically inserted in a pre-planned manner as part of a MISS procedure. In some aspects, lenticular array-based tracking systems may be attached to the tips of cannulae to extrapolate screw locations deep within the tissue.

### Computing Systems and Devices

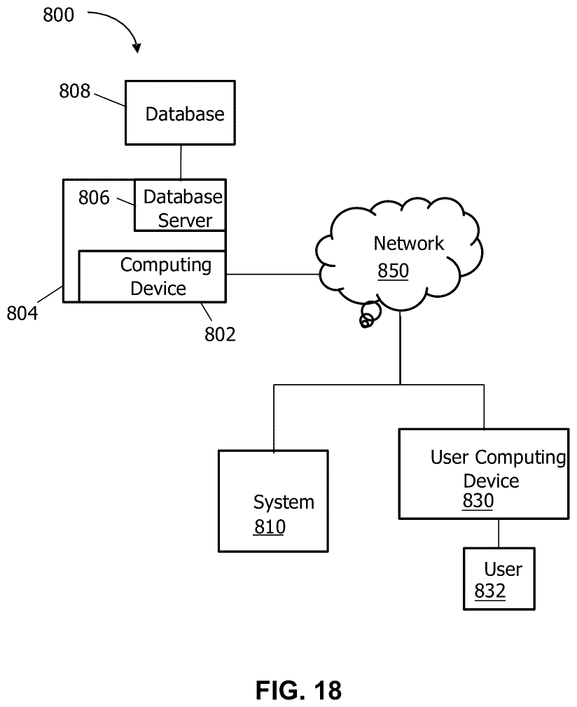

FIG. 18 depicts a simplified block diagram of a computing device 800 for implementing the methods described herein. As illustrated in FIG. 18, the computing device 800 may be configured to implement at least a portion of the tasks associated with the disclosed method using the system. The computer system 800 may include a computing device 802. In one aspect, the computing device 802 is part of a server system 804, which also includes a database server 806. The computing device 802 is in communication with database 808 through the database server 806. The computing device 802 is communicably coupled to the system 810 and a user computing device 830 through a network 850. The network 850 may be any network that allows local area or wide area communication between the devices. For example, the network 850 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 830 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

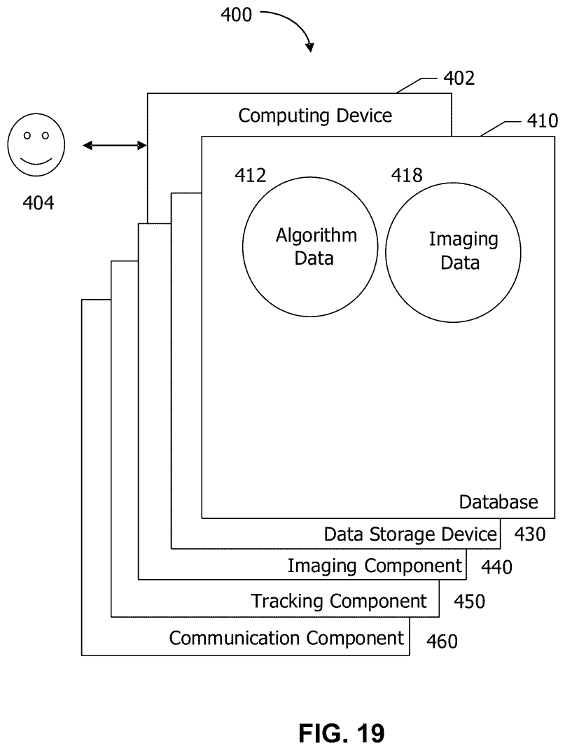

In other aspects, the computing device 802 is configured to perform a plurality of tasks associated with the method of tracking bone screws as described herein. FIG. 19 depicts a component configuration 400 of a computing device 402, which includes a database 410 along with other related computing components. In some aspects, computing device 402 is similar to computing device 802 (shown in FIG. 18). A user 404 may access components of the computing device 402. In some aspects, database 420 is similar to database 808 (shown in FIG. 18).

In one aspect, database 410 includes imaging data 418 and algorithm data 412. Non-limiting examples of suitable imaging data 418 may include images of the surgical region including one or more optical markers as described above. Non-limiting examples of suitable algorithm data 412 include any values of equations or parameters defining the transformation of the position and orientation of pixels corresponding to an optical marker to a global position and orientation of a bone screw as described herein.

The computing device 402 also includes a number of components that perform specific tasks. In an exemplary aspect, the computing device 402 includes a data storage device 430, an imaging component 440, a tracking component 450, and a communication component 460. The data storage device 430 is configured to store data received or generated by the computing device 402, such as any of the data stored in database 410 or any outputs of processes implemented by any component of the computing device 402.

The imaging component 440 is configured to facilitate any tasks associated with obtaining images of the surgical region as well as extracting pixel positions and hues corresponding to the optical markers within the image. The tracking component 440 is configured to facilitate any tasks associated with the transformation of the position and orientation of pixels corresponding to an optical marker to a global position and orientation of a bone screw as described herein. The communication component 460 is configured to enable communications between the computing device 402 and other devices (e.g. user computing device 830 and system 810, shown in FIG. 18 over a network, such as a network 850 (shown in FIG. 18), or a plurality of network connections using predefined network protocols such as TCP/IP (Transmission Control Protocol/Internet Protocol).

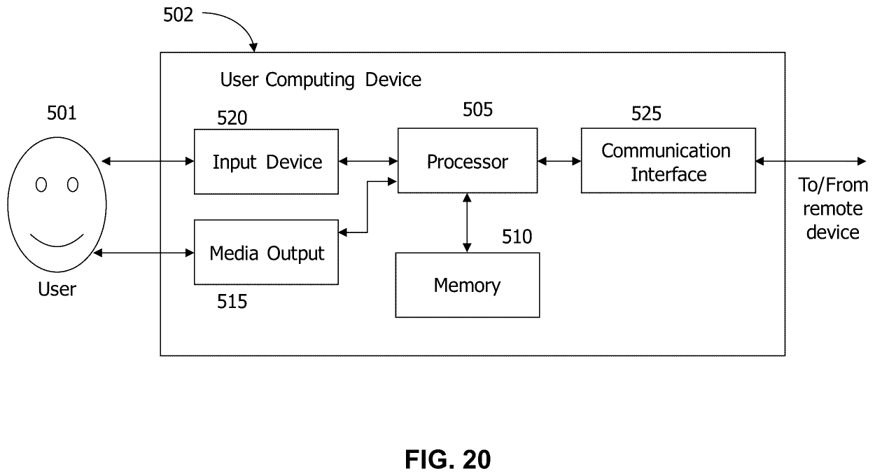

FIG. 20 depicts a configuration of a remote or user computing device 502, such as the user computing device 830 (shown in FIG. 18). The computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile Communications (GSM, 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

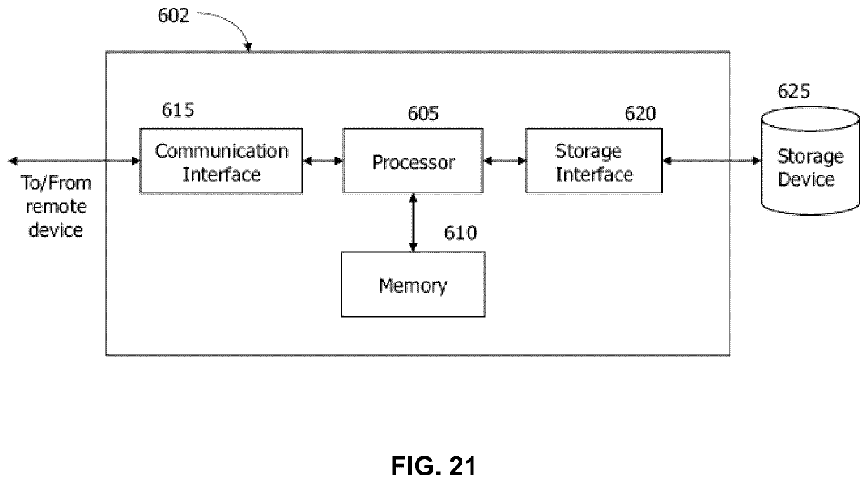

FIG. 21 illustrates an example configuration of a server system 602. Server system 602 may include, but is not limited to, database server 806 and computing device 802 (both shown in FIG. 18). In some aspects, server system 602 is similar to server system 804 (shown in FIG. 18). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 830 (shown in FIG. 18) or another server system 602. For example, communication interface 615 may receive requests from user computing device 830 via a network 850 (shown in FIG. 18).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network-attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIGS. 20) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for the storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Development and Assessment of Hue Response Function for Lenticular Markers

To generate and assess a hue response function (HRF) for the lenticular markers used in the pedicle screw markers disclosed herein, the following experiments were conducted.

A 1.1 by 1.1 cm lenticular marker was constructed using two lenticular array segments with perpendicular major axes. Each lenticular array segment measured 1.1 by 0.55 cm. Photographs of the marker were taken using a smartphone camera (iPhone 7) mounted on a 170 cm long (108 cm diameter) circular track. Photographs were taken at 2.5-degree intervals ranging from 45° to 135°. The lighting in the photography environment consisted of ambient light from ceiling lights as well as the camera flash from the camera. All angles were measured using a protractor and string. The camera was oriented using a right-angle measuring device and an application on the smartphone (bubble level app). After taking all photographs for the first array of interest, the marker was rotated 90° and placed in the same position to obtain photographs for the second array of interest.

For each angle, the image was cropped to isolate the array of interest, the image was converted from the RGB color space to the HSV color space, and the average hue was calculated. The average hue was mapped to its corresponding angle in a hue dictionary for each array. Once the hue dictionaries for each array were established, the relationship between average hue vs. orientation was discovered to be best represented by a 2-term Fourier series using the MATLAB curve-fitting toolbox, and a curve was calculated to best fit the hue-orientation relationship. The hue response function was then calculated according to this curve.

To test the accuracy of the hue response function, photographs of the entire marker were taken at known angles every 1.25° from 65° to 71.25° along the camera track, and the orientation of the camera relative to the array was estimated using the HRF. The HRF for a lenticular array varies depending on lighting, lenticule size, and the color gradients behind them; it was therefore essential to test the HRF for the lenticular marker in the same environment used for calibration.

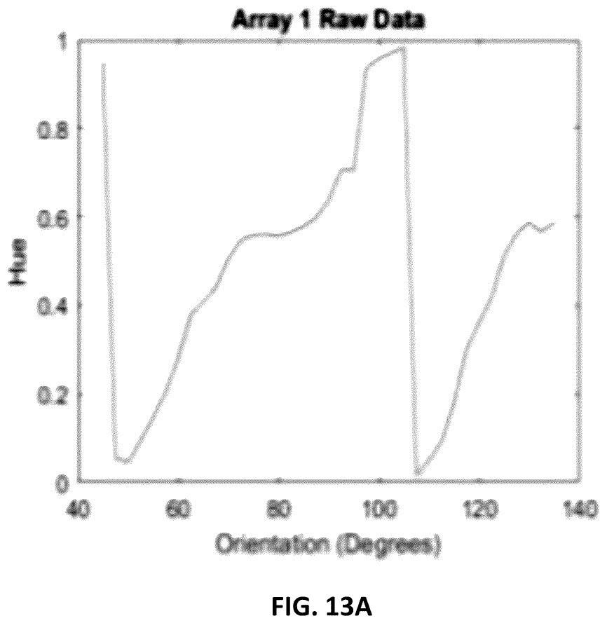

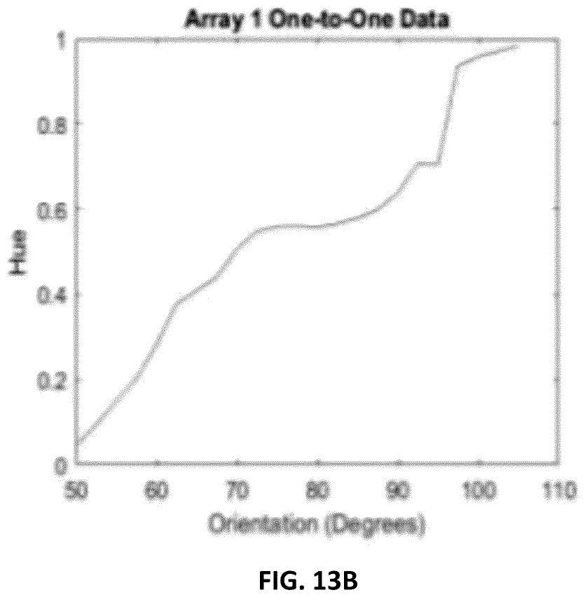

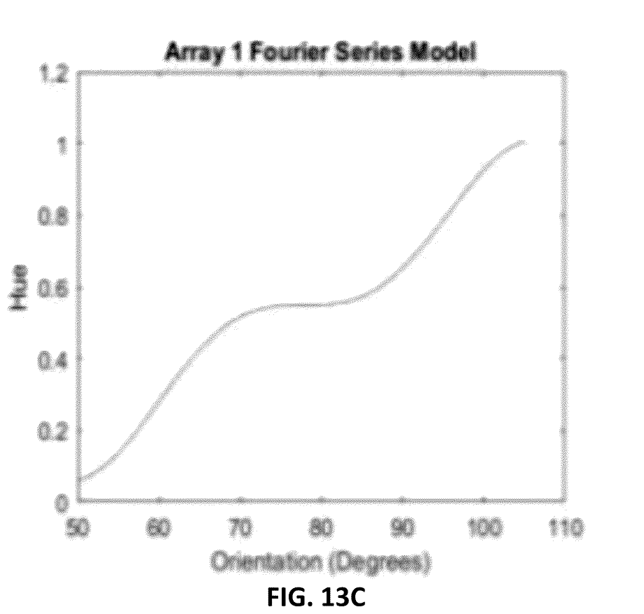

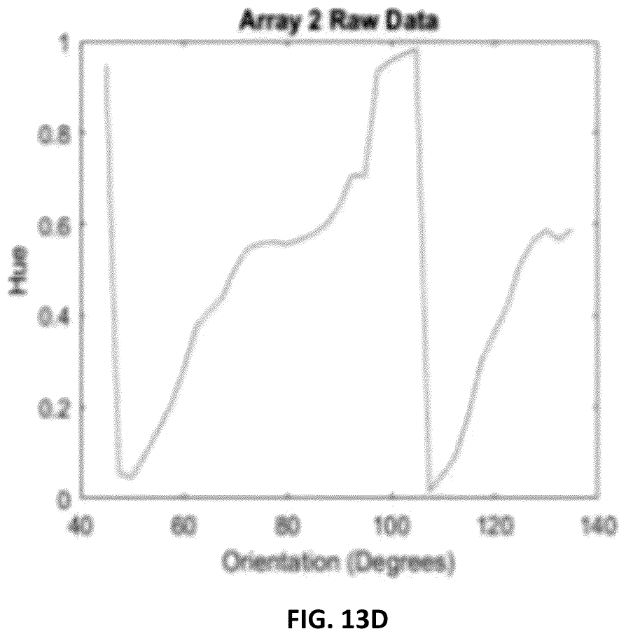

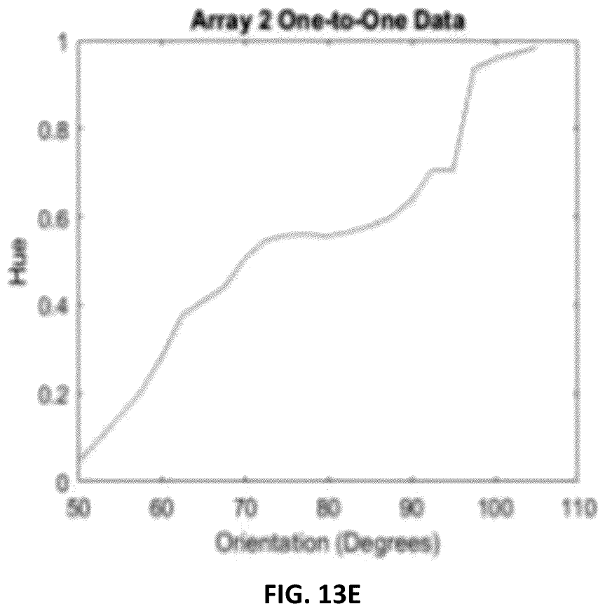

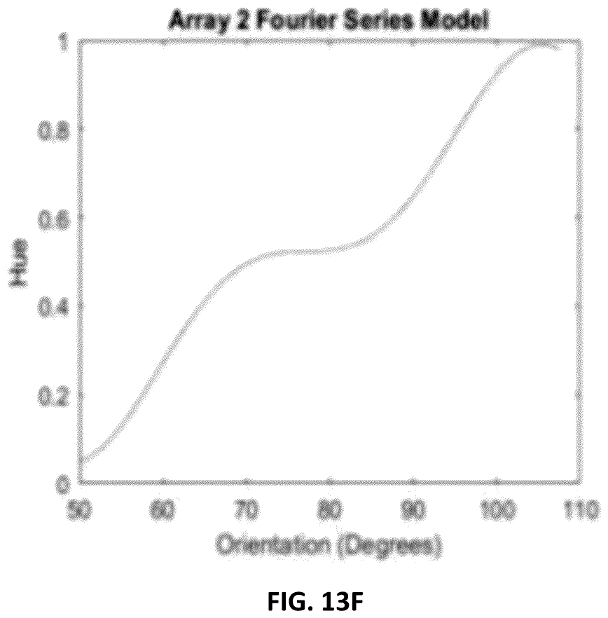

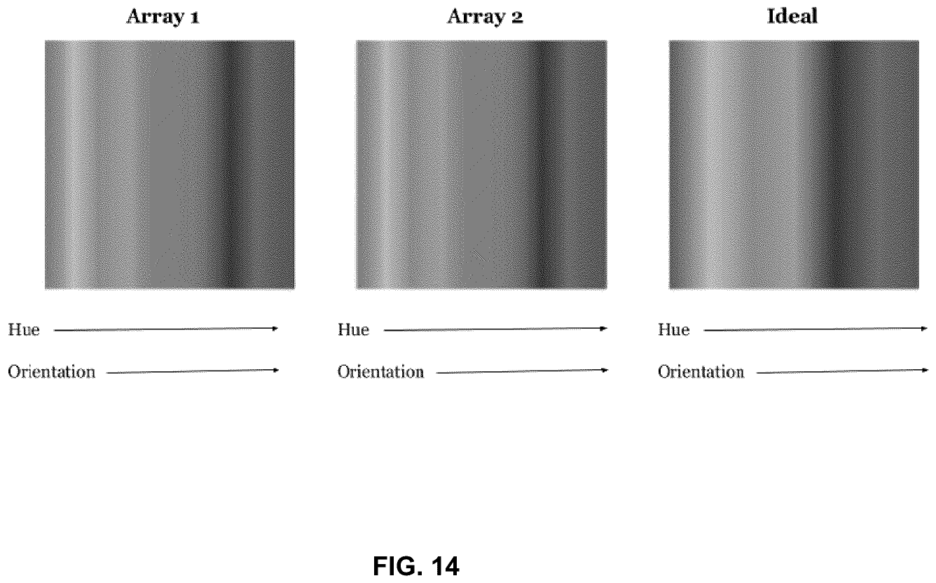

The observed relationship for the lenticular arrays being tested was periodic (see FIGS. 7, 13A, and 13D), and some hues were repeated at different angles (see FIG. 6). This was problematic because the lenticular calibration method requires a unique hue to determine a unique orientation.

To address this issue during testing, only angles ranging from 50° to 105° were considered because this range exhibited an approximately one-to-one relationship between hue and orientation. Raw data for angles 45° through 135° are shown in FIGS. 13A and 13D. Raw data for the one-to-one regions ranging from 50° through 105° are shown in FIGS. 13B and 13E. Fourier series models for the one-to-one hue-orientation relationship are shown in FIGS. 13C and 13F.

The relationships shown in FIGS. 13C and 13F were approximately one-to-one, making orientation estimation using this hue-orientation relationship possible.

Once the HRF fit was generated, photographs were taken every 1.25° from 65° to 71.25° relative to the major axis, beginning from the plane of the marker. The results of the back-calculation of the camera angle relative to the marker for each photograph are shown in Table 1 below.

TABLE 1
Camera Angles Back-Calculated from Marker Images

| Measured Angle | | Calculated Angle | | Error | |
|---|---|---|---|---|---|
| degrees | radians | Degrees | radians | degrees | radians |
| 65 | 1.134 | 69.35 | 1.211 | 4.35 | 0.077 |
| 66.25 | 1.156 | 69.90 | 1.220 | 3.65 | 0.064 |
| 67.5 | 1.178 | 70.99 | 1.239 | 3.49 | 0.061 |
| 68.75 | 1.200 | 71.50 | 1.248 | 2.75 | 0.048 |
| 70 | 1.221 | 71.91 | 1.255 | 1.91 | 0.034 |
| 71.25 | 1.245 | 74.82 | 1.306 | 3.03 | 0.061 |

All of the observed errors were within 5°.

In future experiments, a motorized camera track with a fixed speed may be used to obtain video images of the lenticular array and relate each frame to orientation for more accurate angle measurements. A camera mount to hold the camera perfectly tangential to the arc may also be used to improve the accuracy of angle measurements. In addition, a perfectly linear hue gradient placed behind the lenticular array during calibration will more practically address the issue of generating a one-to-one HRF.

### Example 2: Design and Fabrication of Pedicle Screw Extensions

To design and fabricate the pedicle screw extensions used in the pedicle screw markers disclosed herein, the following experiments were conducted.

PLA plastic was used to construct prototypes of pedicle screw extensions that would be safe, easy-to-use, reliable, and as universally applicable as possible. The design of the first prototype focused on the driveability of the screw through the extension, integration with existing technology and proprietary threadings, creation of a large surface area for markers, and monoaxial constraint of polyaxial screws.

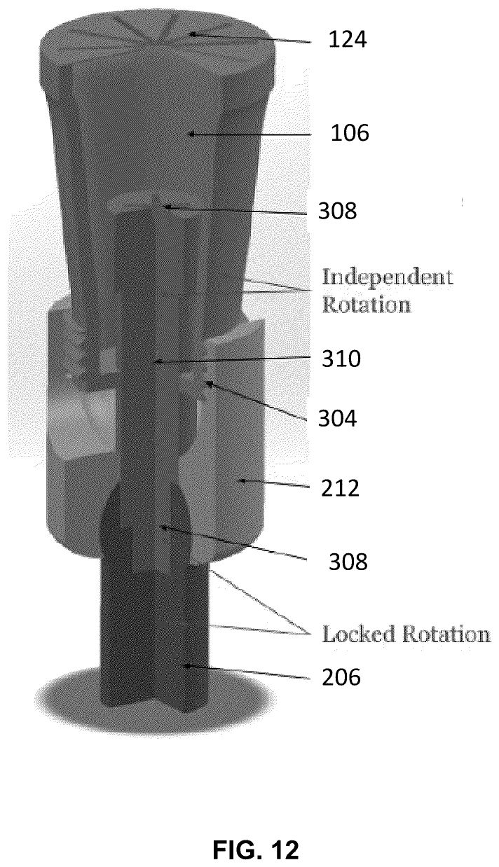

This design is shown in FIG. 12 and consists of a lofted marker attachment and a driving pin coaxially inserted into the loft. The lofted attachment possesses threads matching proprietary threading from the relevant screw tulip brand and size; the screw used in this study was a two-times scale, 3D print modeled after the Stryker EVEREST Screw (Catalog #2911-06545, (GUD) Primary Device Identifier Number: 10888857042339). The driving pin integrates with the geometry of the EVEREST Screw tulip while using its proprietary driver shape and size to set into the shank drive, thereby constraining the polyaxial motion of the tulip to monoaxial and interlacing any rotation of the driving pin with that of the shank. This prototype is static and cannot be manipulated or bent into free space without breakage, revealing a major flaw when applied in the lumbar spine, where screws may be inserted at an angle and therefore not trackable with a static extension due to visual and physical constraints created by walls of fat, tissue, or muscle.

The final design uses a hinge mechanism to expand its applications for the lumbar spine while maintaining its usefulness in the thoracic spine. Pedicle screws in the lumbar spine are inserted at a lateral to medial orientation, meaning that any statically coaxial extension protruding from the tulip would be more likely to conflict with existing walls of muscle, fat, and tissue when compared to screws in the thoracic spine. This emphasizes the need for at least two degrees of rotational freedom in the extensions themselves, such that the extension can be rotated about its base and therefore redirected into more open and visible spaces for tracking. Additionally, the inclusion of rotational degrees of freedom necessitates a method for tracking the angle of the marker relative to the tulip to fully define tulip pose.

To address the increased need for rotational freedom, the final design (shown in FIGS. 3B and 4) leverages the pedicle screw tulip's ability to rotate to provide one rotational degree of freedom by using a simple cylinder to interface with the shank drive, thereby retaining the monoaxial configuration of the screw while still allowing independent rotation of the tulip and extension from the shank. The second degree of rotational freedom is enabled by the hinge 108 between the extension arm 104 and extension base 102 as shown in FIG. 3B. Locking pegs are located on the outside of the marker arm to interlock with their counterpart holes in the extension base on a circular track, allowing the user to rotate the arm in increments of 30° ; these increments allow the markers to be roughly parallel to the image plane while still maintaining a precise and known polar angle. The chosen angle of the hinge (polar angle) is tracked by the user, while the azimuthal angle is calculated by the rotation of the marker about the into-plane camera axis.

As an extra precaution against obstruction by walls of muscle, fat, or tissue, the marker arm can be attached to the extension base before or after it is threaded into the tulip. There are two entry slots located at 0° and 90° polar angles to ensure that the extension arm 104 can be attached without difficulty, as seen in FIG. 3B.

What is claimed is:

1. An extension for a bone screw configured to track a position and orientation of the bone screw, the extension comprising:
a. an extension base comprising an attachment fitting configured to couple to the bone screw and further comprising a hinge fitting;
b. a marker arm comprising a hinge end and a marker support at opposite ends of the marker arm, wherein the hinge end is coupled to the hinge fitting to form a hinged joint; and
c. an optical marker attached to the marker support, the optical marker comprising a first and second lenticular array comprising first and second major axes, respectively, wherein the first and second lenticular arrays are positioned in a coplanar arrangement on the marker support and the first and second major axes are oriented perpendicularly.

2. The extension of claim 1, wherein the attachment fitting comprises a threaded end configured to mesh with a corresponding threaded tulip head of the bone screw.

3. The extension of claim 2, wherein the attachment fitting further comprises a drive fitting projecting downward from the attachment fitting, the drive fitting ending in a drive end configured to mesh with a shank head of the bone screw.

4. The extension of claim 3, wherein the drive fitting is further configured to constrain the tulip head of the bone screw to a monoaxial configuration.

5. The extension of claim 2, wherein the tulip head of the bone screw is constrained to rotate in an azimuthal rotation about a screw axis of the bone screw.

6. The extension of claim 1, wherein the hinged joint constrains the marker arm to rotate in a polar rotation relative to the screw axis.

7. The extension of claim 1, wherein the hinge fitting and hinge end of the marker are further comprised of interlocking features to selectively lock the polar rotation of the marker arm to one of at least two predetermined polar angles.

8. The extension of claim 7, wherein the at least two predetermined polar angles are selected from 0°, 15°, 30°, 45°, 60°, and 90°, wherein 0° corresponds to the marker arm projecting upward and parallel to the screw axis and 90° corresponds to the marker arm oriented perpendicular to the screw axis.

9. The extension of claim 8, wherein the predetermined polar angles are selected from 0°, 30°, 60°, and 90°.

10. The extension of claim 1, wherein the bone screw is selected from a monoaxial screw, a polyaxial screw, a uniaxial screw, a uniplanar pedicle screw, and a reduction iliac screw.

11. The extension of claim 1, wherein the first and second lenticular arrays are each configured to display a hue that varies with an orientation of each lenticular array relative to a viewer or image recording device.

12. A trackable bone screw, comprising an extension for a bone screw configured to track a position and orientation of the bone screw. the extension coupled to a bone screw, the extension comprising:
a. an extension base comprising an attachment fitting configured to couple to the bone screw and further comprising a hinge fitting;
b. a marker arm comprising a hinge end and a marker support at opposite ends of the marker arm, wherein the hinge end is coupled to the hinge fitting to form a hinged joint; and
c. an optical marker attached to the marker support, the optical marker comprising a first and second lenticular array comprising first and second major axes, respectively, wherein the first and second lenticular arrays are positioned in a coplanar arrangement on the marker support and the first and second major axes are oriented perpendicularly.

13. The trackable bone screw of claim 12, wherein the bone screw is selected from a monoaxial screw, a polyaxial screw, a uniaxial screw, a uniplanar pedicle screw, and a reduction iliac screw.

14. A system for tracking a position and orientation of at least one trackable bone screw, the system comprising a computing device, the computing device comprising at least one processor configured to:
a. receive an image of a surgical region, the image comprising a plurality of pixels, each pixel comprising a pixel position and a hue, wherein at least one pixel portion of the plurality of pixels corresponds to an optical marker of a trackable bone screw of any preceding claim;
b. for each of the at least one trackable bone screws:
i. extract, using the computing device, a first pixel portion of the plurality of pixels corresponding to the optical marker;
ii. transform the first pixel portion into a global position and orientation of the optical marker based on the pixel positions and hues of the first pixel portion;
iii. determine a relative displacement of the optical marker from a screw head of the bone screw based on a polar angle, an azimuth angle, and a length of the marker arm of the extension of the bone screw; and
iv. determine the global position and orientation of the bone screw by combining the relative displacement of the optical marker from the screw head with the global position and orientation of the optical marker.

15. The system of claim 14, wherein the first pixel portion is transformed into the global position and orientation of the optical marker based on a pinhole camera model subject to a group of constraints corresponding to the pixel positions and hues of the first pixel portion.

16. The system of claim 15, wherein the global position of the first pixel group comprises a rotation matrix R and a translation matrix T defining the rotation and translation of the pixels within a camera coordinate system to corresponding objects in a global coordinate system.

17. The system of claim 16, wherein the rotation matrix R is obtained by solving the equations:
R\vec{n}ₕᵤₑ_{₁}·\vec{r}₁=0 Eqn. (8);
R\vec{n}ₕᵤₑ_{₂}·\vec{r}₂=0 Eqn. (9); and
R(C₂-C₁)×(\vec{r}₁×r₂)=0 Eqn. (10),
wherein:
\vec{n}ₕᵤₑ_{₁} and \vec{n}ₕᵤₑ_{₂} are vectors based on the pixel hues corresponding to the first and second lenticular arrays, respectively;
\vec{r}₁ and \vec{n}ₕᵤₑ_{₂} are rays passing from the origin of a camera origin system through portions of the first pixel group corresponding to the first and second lenticular arrays, respectively; and
(C₂-C₁) is the global displacement distance between the first and second lenticular arrays.

18. The system of claim 16, wherein the translation matrix T is obtained by solving the equations:
(RC₁+T)·\vec{r}₁=\vec{0} Eqn. (12)
(RC₂+T)·\vec{r}₂=\vec{0} Eqn. (13)
wherein C₁ and C₂ are the global positions of the first and second lenticular arrays, respectively.

19. The system of claim 18, wherein the wherein \vec{n}ₕᵤₑ_{₁} and \vec{n}ₕᵤₑ_{₁} are obtained based on the hues of the portions of the first pixel group corresponding to the first and second lenticular arrays, respectively, using at least one predetermined hue response function.

20. The system of claim 14, further comprising an imaging device operatively coupled to the computing device, wherein the imaging device is configured to obtain the image of the surgical region.
